(19) United States
(12) Patent Application Publication
(10) Pub. No.: US 2026/0071225 A1
(43) Pub. Date: Mar. 12, 2026

(54) BACTERIUM WITH REDUCED SENSITIVITY TO BACTERIOPHAGE

(71) Applicant: DUPONT NUTRITION BIOSCIENCES APS, Kongens Lyngby (DK)

(72) Inventors: Jennifer Seiler, New Century, KS (US); Anne M. Millen, New Century, KS (US); Dennis Romero, New Century, KS (US)

(21) Appl. No.: 18/254,476

(22) PCT Filed: Nov. 24, 2021
(86) PCT No.: PCT/EP2021/082750

§ 371 Date: May 25, 2023

Related U.S. Application Data
(60) Provisional application No. 63/118,315, filed on Nov. 25, 2020.

(51) Int. Cl. C12N 15/74; A23C 9/123; A23K 10/12; C07K 14/315; C12N 9/36; C12R 1/46

(52) U.S. Cl. CPC C12N 15/746; A23C 9/123; A23K 10/12; C07K 14/315; C12N 9/2462; C12R 2001/46

(57) ABSTRACT

A bacterial strain which has been which has been modified to reduce the activity and/or expression of autolysin, wherein said modified strain has reduced sensitivity to bacteriophage, bacterial compositions comprising the bacterial strain and use of the strain or composition in manufacture of a food or feed product.

### CROSS-REFERENCE TO RELATED PATENT APPLICATIONS

This specification claims priority under 35 USC § 371 as a national phase of International Patent Application PCT/EP2021/082750 (filed Nov. 24, 2021; and published Jun. 2, 2022 as Int'l Publ. No. WO2022/112284), which, in turn, claims priority to U.S. Provisional Patent Application No. 63/118,315 (filed Nov. 25, 2020). The entire text of each of the above-referenced patent applications is incorporated by reference into this specification for all purposes.

### INCORPORATION BY REFERENCE OF THE SEQUENCE LISTING

The present application is being filed along with a Sequence Listing in electronic format. The Sequence Listing is provided as a file entitled NB41696-WO-PCT_seq_list_ST25.txt, created Mar. 27, 2023 which is 11,607 bytes in size. The information in the electronic format of the Sequence Listing is incorporated by reference in its entirety.

### FIELD OF THE INVENTION

The present invention relates to bacterial strains with reduced sensitivity to bacteriophages. The invention also relates to compositions comprising at least one strain of the invention, and to the use of this strain or composition to manufacture a food, such as a dairy product or to manufacture a feed.

### BACKGROUND TO THE INVENTION

Bacterial starter cultures are used extensively in the food industry in the manufacture of fermented products including milk products (such as yoghurt, butter and cheese), meat products, bakery products, wine and vegetable products.

The attack of bacterial cultures by virulent bacterial viruses (bacteriophage, or phage) and multiplication is considered to be one of the major problems with the industrial use of bacterial cultures, leading to production failures of bacterial cultures. Due to the nature of commercial fermentations, virulent phage are commonly found in the industrial environment. If left uncontrolled, bacteriophage will interfere with the fermentation resulting in a disruption of the process and loss of product quality. Bacteriophages have been found for many of the bacterial strains used in the industry and there are numerous different types of phages with varying infection mechanisms. New strains of bacteriophages continue to emerge.

Typically, the lytic infection cycle of bacteriophages involves phage adsorption to the bacterial host cell surface, injection of phage DNA to the cell, phage DNA replication, phage protein expression, phage assembly and host bacterial cell lysis to release the assembled phage. Bacterial phage resistance mechanisms depend upon host factors involved in one or more steps of the lytic cycle of phage replication and are generally classified based on the step of the infectious cycle they inhibit.

Strategies used in the industry to minimise bacteriophage infection, and thus failure of a bacterial culture, are not fully effective. Such strategies include the use of mixed starter cultures to ensure that a certain level of resistance to phage attack is present. In addition, rotation of selected bacterial strains which are sensitive to different bacteriophages is used. However, rapid replacement of the bacterial strain with a resistant strain following the emergence of a new phage is usually not possible. Therefore, it has not yet been possible to eliminate phage contamination in the food industry.

There is a continuing need in the art to provide improved bacterial strains for use in the food/feed industry—such as bacterial strains that are phage resistant. There is therefore a need to identify host factors involved in phage replication in lactic acid bacteria, for example in the genera Streptococcus.

### SUMMARY OF THE INVENTION

The present inventors have surprisingly found that a host gene coding for autolysin is involved in phage replication and is necessary for bacteriophage, such as P335-like phage D6867, to complete its lytic cycle. A mutation in the autolysin gene provides the host cell (such as a Lactic acid bacterium) with resistance to bacteriophage, such as a P335-like phage e.g. D6867. Complementation of the mutated strains with the autolysin gene of a sensitive strain results in restoration of the phage-sensitivity phenotype. The inventors have also demonstrated that bacteria comprising a mutation in autolysin maintain commercially acceptable milk acidification kinetics, making them suitable for use in methods of producing food or feed, such a fermented products e.g. fermented dairy products.

In one aspect, the present invention provides a bacterial strain which has been modified to reduce the activity and/or expression of autolysin, wherein said modified strain has reduced sensitivity to bacteriophage (such as at least one P335-like phage, e.g. D6867).

Suitably, the modification may render autolysin partially or completely non-functional with respect to phage infection.

Suitably, the modification may be a stop codon, an insertion, a deletion or a mutation.

Suitably, the modification may be introduced into a nucleic acid sequence which encodes said autolysin, or in to a regulatory region (such as a promoter or enhancer) which contributes to controlling the expression of said autolysin.

Suitably, the bacterial strain may be modified to comprise an autolysin^{R} allele encoding an autolysin^{R} protein, wherein said autolysin^{R} allele reduces the sensitivity to phage D6867 of a Lactococcus lactis SL12699 derivative strain, said SL12699 derivative strain being a Lactococcus lactis SL12699 strain into which its autolysin allele has been replaced by said autolysin^{R} allele, and wherein sensitivity to phage D6867 is determined by Efficiency of Plaquing (EOP) Assay I.

Suitably, the bacterial stain may be homozygous for the modification.

In one aspect, the present invention provides a method for increasing (e.g. conferring or increasing) the resistance of a bacterial strain to bacteriophage (such as at least one P335-like phage, e.g. D6867) comprising modifying said bacterial strain by decreasing the activity and or expression of autolysin.

In one aspect, the present invention provides a method for preparing at least one bacteriophage resistant variant strain (such as resistance to at least one P335-like phage, e.g. D6867), comprising modifying a parental bacterial strain to decrease the activity and or expression of autolysin.

Suitably, a method according to the present invention may comprise
a) providing a bacterial strain (such as a Lactic acid bacterial strain of the Lactococcus genus) sensitive to at least one bacteriophage (such as a P335-like phage, e.g. D6867);
b) modifying said bacterial strain to reduce the activity and/or expression of autolysin; and
c) recovering the strain(s) having reduced sensitivity to at least one bacteriophage (such as a P335-like phage, e.g. D6867), optionally wherein sensitivity to at least one bacteriophage (such as a P335-like phage, e.g. D6867) is determined by EOP Assay I.

In one aspect, the present invention provides a bacterial strain obtainable or obtained by a method according to the present invention.

In one aspect, the present invention provides a polynucleotide encoding a mutated autolysin protein (autolysin^{R} protein) as defined herein.

Suitably, a polynucleotide according to the present invention may reduce the sensitivity to phage D6867 of a Lactococcus lactis SL12699 derivative strain, said SL12699 derivative strain being a Lactococcus lactis SL12699 strain into which its autolysin allele has been replaced by said autolysin^{R} allele, and wherein sensitivity to phage D6867 is determined by Efficiency of Plaquing (EOP) Assay I.

In one aspect, the present invention provides a construct or a vector comprising the polynucleotide according to the present invention.

In one aspect, the present invention provides the use of a polynucleotide according to the present invention, or of a construct or vector according to the present invention, to reduce the sensitivity of a bacterial strain to at least one bacteriophage (such as a P335-like phage, e.g. D6867).

In any aspect of the present invention, the reduction of sensitivity to phage (such as to a P335-like phage, e.g. D6867, e.g. D6867) may be characterized by an EOP reduction of at least 4 log, of at least 5 log or of at least 6 log.

In any aspect of the present invention, the modification may encode an autolysin^{R} protein comprising an amino acid suppression, an amino acid addition, an amino acid substitution or an amino acid suppression and addition, relative to an autolysin protein selected from the group consisting of:
a) an autolysin protein having an amino acid sequence as defined in SEQ ID NO:2;
b) an autolysin variant protein comprising an amino acid sequence having at least 80% identity with SEQ ID NO:2 encoded by a autolysin allele, which does not reduce the EOP of phage D6867 on a Lactococcus lactis SL12699 derivative strain, said SL12699 derivative strain being a Lactococcus lactis SL12699 strain into which its autolysin allele has been replaced by the autolysin allele encoding said autolysin variant protein and wherein sensitivity to phage D6867 is determined by Efficiency of Plaquing (EOP) Assay I; and
c) an autolysin variant protein comprising an amino acid sequence having at least 80% identity with SEQ ID NO:2 encoded by a autolysin allele, which reduces the EOP of phage D6867 on a Lactococcus lactis SL12699 derivative strain, of less than 3 log, said D6867 derivative strain being a Lactococcus lactis SL12699 strain into which its autolysin allele has been replaced by the autolysin allele encoding said autolysin variant protein and wherein sensitivity to phage DT1 is determined by Efficiency of Plaquing (EOP) Assay I.
d) a homologue of SEQ ID NO:2 encoded by a autolysin allele, which does not reduce the EOP of phage D6867 on a Lactococcus lactis SL12699 derivative strain, said SL12699 derivative strain being a Lactococcus lactis SL12699 strain into which its autolysin allele has been replaced by the autolysin allele encoding said autolysin variant protein and wherein sensitivity to phage D6867 is determined by Efficiency of Plaquing (EOP) Assay I; and
e) a homologue of SEQ ID NO:2 encoded by a autolysin allele, which reduces the EOP of phage D6867 on a Lactococcus lactis SL12699 derivative strain, of less than 3 log, said D6867 derivative strain being a Lactococcus lactis SL12699 strain into which its autolysin allele has been replaced by the autolysin allele encoding said autolysin variant protein and wherein sensitivity to phage DT1 is determined by Efficiency of Plaquing (EOP) Assay I.

In any aspect of the present invention, the modification may result in truncation of the YjdB domain of autolysin.

In any aspect of the present invention, the truncated autolysin protein may be the result of a deletion and/or an insertion in the autolysin allele.

In any aspect of the present invention, the truncated autolysin protein may be the result of a nucleotide mutation leading to a STOP codon.

In any aspect of the present invention, the bacterial strain may be additionally mutated in its pip gene and have resistance to c2-type c2 phages.

In any aspect of the present invention, the bacterial strain may be additionally mutated in its yjaE gene and have resistance to bil67-type c2 phages.

In any aspect of the present invention, the bacterial strain may be a lactic acid strain, suitably the lactic acid bacterial strain may be selected from the genera Lactococcus, Streptococcus, Lactobacillus, Leuconostoc, Pediococcus and Bifidobacterium, suitably, the strain may be of the Lactococcus genus, suitably said strain may be a Lactococcus sp., such as a Lactococcus lactis species, Lactococcus lactis subsp. cremoris, Lactococcus lactis subsp. lactis or Lactococcus lactis subsp. lactis biovar. Diacetylactis.

In one aspect, the present invention provides a bacterial composition comprising a bacterial strain according to the present invention. The bacterial composition may optionally further comprise one or more further lactic acid bacteria selected from the group consisting of Lactococcus, Streptococcus, Lactobacillus, Leuconostoc, Pediococcus, Enterococcus, Oenococcus and Bifidobacterium.

In one aspect the present invention provides a food or feed product comprising a bacterial strain according to the present invention, or a bacterial composition according to the present invention. The product may be a dairy, meat or cereal food or feed product, more particularly a fermented dairy product.

Suitably, the dairy food or feed product may be a fermented dairy product, such as a fermented milk, yoghurt, cream, matured cream, cheese, fromage frais, a milk beverage, a processed cheese, a cream dessert, a cottage cheese or infant milk.

In one aspect, the present invention provides a method for manufacturing a fermented product, comprising:
a) inoculating a substrate, preferably a milk substrate, with a bacterial strain according to the present invention, or a bacterial composition according to the present invention; and
b) fermenting the inoculated substrate obtained from step a) to obtain a fermented product, preferably a fermented dairy product.

In one aspect, the present invention provides the use of a bacterial strain according to the present invention, or a bacterial composition according to the present invention, to manufacture a food or feed product. The product may be a fermented food product. The product may be a fermented dairy product.

### BRIEF DESCRIPTION OF THE FIGURES

FIG. 1 shows a comparison of the three mutations found in the autolysin protein in phage-resistant variants of SL12699. The number line represents the amino acid sequence of the autolysin protein and the boxes within those bars indicate where the protein is no longer being transcribed. The locations of the various domains are shown below the altered proteins.

FIG. 2 compares the milk acidification activity of autolysin mutant SL12852 to parent SL12699.

FIG. 3 shows the milk acidification activity of SL12852 with various multiplicity of infection values (MOIs) of phage D6867 added.

FIG. 4 shows the milk acidification activity of SL12699 with various MOIs of phage D6867 added. This testing with the phage-sensitive parental strain serves as the control for the testing in FIG. 3.

### DETAILED DESCRIPTION OF THE INVENTION

### General Definitions

Unless defined otherwise, all technical and scientific terms used herein have the same meaning as commonly understood by one of ordinary skill in the art to which this disclosure belongs.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range.

The headings provided herein are not limitations of the various aspects or embodiments of this disclosure which can be had by reference to the specification as a whole. Accordingly, the terms defined immediately below are more fully defined by reference to the specification as a whole.

As used herein, the term “polynucleotide” it is synonymous with the term “nucleotide sequence” and/or the term “nucleic acid sequence”. Unless otherwise indicated, any nucleic acid sequences are written left to right in 5′ to 3′ orientation.

The term “protein”, as used herein, includes proteins, polypeptides, and peptides. As used herein, the term “amino acid sequence” is synonymous with the term “polypeptide” and/or the term “protein”. In the present disclosure and claims, the name of the amino acid, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code. Unless otherwise indicated, any amino acid sequences are written left to right in amino to carboxy orientation.

In the present invention, a specific numbering of amino acid residue positions in the autolysin used in the present invention may be employed. By alignment of the amino acid sequence of a sample autolysin with the autolysin of SEQ ID NO: 2 it is possible to assign a number to an amino acid residue position in said sample autolysin which corresponds with the amino acid residue position or numbering of the amino acid sequence shown in SEQ ID NO: 2 of the present invention.

Other definitions of terms may appear throughout the specification. Before the exemplary embodiments are described in more detail, it is to understand that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of’ as used herein are synonymous with “including”, “includes” or “containing”, “contains”, and are inclusive or open-ended and do not exclude additional, non-recited members, elements or method steps. The terms “comprising”, “comprises” and “comprised of’ also include the term “consisting of’.

The publications discussed herein are provided solely for their disclosure prior to the filing date of the present application. Nothing herein is to be construed as an admission that such publications constitute prior art to the claims appended hereto.

### Autolysin

The present inventors have surprisingly found that a host gene coding for autolysin is a bacterial host factor involved in bacteriophage multiplication in bacterial strains, such as lactic acid bacteria e.g. from the Lactococcus genus.

In one aspect, the present invention provides a method for increasing (e.g. conferring or increasing) the resistance of a bacterial strain to a bacteriophage (such as at least one P335-like phage) comprising modifying said bacterial strain by decreasing (or inhibiting) the activity and or expression of autolysin.

The term “increased resistance to a bacteriophage” denotes that the bacteria strain when tested in a plaque assay, such as the assay “Efficiency of Plaquing Assay I” described below have an improved phage resistance to at least one phage.

In one aspect, the present invention provides a method for preparing at least one bacteriophage resistant variant strain (such as resistance to at least one P335-like phage), comprising modifying a parental bacterial strain to decrease (or inhibit) the activity and or expression of autolysin. The bacterial resistant variant strain has increased phage resistance to at least one phage when compared with the parental strain.

In other words, a modification has been introduced to the bacterial strain which reduces (or inhibits) the activity and/or expression of autolysin. Suitably, the activity or expression of said autolysin may be completely eliminated.

The “expression” of autolysin may refer to the level of transcription or translation of autolysin. Gene expression may be measured at the RNA level and, for protein-coding genes, at the protein level. For example, RNA levels can be measured by quantitative reverse transcription polymerase chain reaction (RT-qPCR), reverse transcription polymerase chain reaction (RT-PCR), RNA sequencing (RNA-seq), Northern blotting, DNA microarrays and protein levels can be measure by western blotting, enzyme-linked immunosorbent assay (ELISA) and mass spectrometry.

The “activity” of the autolysin refers to its activity in the context of phage infection.

The modification may render the autolysin partially or completely non-functional with respect to phage infection.

The term “non-functional” as used herein should be understood in the context of the objective of the present invention. The objective is to make a strain where the autolysin protein is less effective than in a corresponding wild-type strain. For instance, by introducing a stop codon, or frame shift insertion in the autolysin gene, which could give a non-functional gene that would could express no autolysin protein, or express a partial length inactive autolysin protein. Alternatively, a mutation could be made in the gene, which e.g. could produce an autolysin protein mutation variant is expressed but which for all herein purposes is not active, One way of measuring the activity of the autolysin protein is to analyse the bacterial strain for increased resistance to a representative phage e.g. a P335-like phage such as D6867. If the bacterial strain has an increased resistance to the representative phage, it is understood that the autolysin protein is at least partially inactive or is completely inactive. In one aspect, the autolysin protein is rendered completely inactive.

The modification may be a stop codon, an insertion (e.g. which causes a frame shift), a deletion or mutation. In other words, the activity and/or expression of autolysin is reduced by the introduction of a stop codon, an insertion (e.g. which causes a frame shift), a deletion or a mutation.

The modification (such as a mutation) may be introduced into a nucleic acid sequence which encodes said autolysin, or in to a regulatory region (such as a promoter or enhancer) which contributes to controlling the expression of said autolysin.

As used herein “autolysin” refers to an endogenous lytic enzyme which breaks down the cell-wall peptidoglycan of the bacteria which produce them. Autolysins are classified as peptidoglycan hydrolases.

Autolysins are involved in cell growth, cell wall metabolism, cell division and separation and peptidoglycan turnover. Some bacteriophage (such as S. pneumoniae) are believed to harness the ubiquitous host autolysin to accomplish optimal host cell lysis.

The autolytic activity of lactic acid bacteria is an important factor in production of dairy products such as in cheese ripening, due to the release of intracellularly-located enzymes into the curd and their action on flavour development. The main consequence of autolysed cells in cheese production is to accelerate the peptidolytic reactions, whilst intact cells are necessary for physiological reactions such as lactose fermentation and oxygen removal.

The nucleotide sequence which encodes SL12699 autolysin is set forth in SEQ ID NO: 1:

(SEQ ID NO: 1)
ATGAAACTGAAAAAAACTCACATTATTTCACTAATACTCTTTTCT
GGATTACTATTTACAAAACCAGTATTAGCTGAAGACACTATTAGT
TCAGATAACACCCCGATGGGGCACTACATTGAGCAAATTCAAACT
CAAGAAACTTCCAAGGACAGTAGTAGTGATACATCATCTACAACT
CCTAATATCAGAGCACGTTCATTTGCAGCAGCTCCAACTGCCAAT
GTTCCCTCTGATCTCAAGTCCGATGATAATACACTGCCAAGAAAA
GATGCTGTAGATATTGCTAGCTATCAATCATGGATGACACAAGCA
GACTTTAATTCTCTTAAGACATCTGGTGTCAAGTCAATCGTTGTT
AAACTAACCGAAGGAACTAATTATACGAACCCATATGCTGCAAAT
CAAATTAAAATGGCACAGAATGCCGGCTTAAATGTTGCAGTTTAT
CACTACGCACGCTTAACAGGAGCCAATTCACAAAGTGATGCAAAT
TCATTAGCCATACAGGAAGCCGCATACTTTGCAAAAGTAGCTAAA
TCTTTTGGATTGTCTAGCAACATTGTAATGATTATGGACTGCGAG
CAACCTTATAGAGATGGCTCGGGAAATATTATCGGACCCAATCCC
ATAACGGTTGATTGGGCAACAGCTGGTGTACAATTTGCTAACACG
CTCAAAGCAAATGGTTATAGCAACACAAAGTTTTATACCTCTGCT
TCATGGATTGGAACAGATACAGCAACTTGTCAAATGAACTATAAT
ACTCTAGGTGGTCTCAAAAATCTTTGGGCTGCTCAATATCTATAT
GGCAAGCCATCTTCTAGTAATCTTCAGAATACGCAATATGGTGCT
TGGCAATATACAAGTCAGATGTATTATCAAGGAACCTCTAACTTG
AAAGCCAATGCTGTTGATACTTCAATTGATTATAGTAATTATTTT
ACCTCTACATCCCCTGTTCCTCCTTCAACTTATACTATAACATTT
AACACAGACGGTGGAACACCAATTGCCAATCAATCGGTTGCAGCT
GGTAGTGTAGTAAGCCAACCAGCAGCTCCTACTAAAGCTGGATTT
AACTTCTCAGGTTGGTATAGTGATTCTTCACTTACTCAAGCTTAT
AACTTCGCAAAAGTAATTACATCTAATACAACCCTTTATGCTAAA
TGGATCCCGATAACATCTCCTTCAATTTCTTACCAAGCCCAAGTC
CAAAATATAGGATGGCAAAAAAATTCCTATAACGGAGAAACTGCT
GGTACAACTGGGTTAGGGTTACGCATGGAGTCACTTAAAATTAGC
CTTATCAACCTTTCAAACGGTCTGACTAATTCAAATAGTCACATT
CAATATCAGGGTTATGTACAAAATATCGGATGGCAAAATCCGGTT
CAAGATGGGACAATTGCTGGAACAGTTGGACAAGGGTTGCGATTT
GAGGCAATAAAAATGAATTTAAGCGGAGAAATAGCAAATCAATAC
GATCTATACTACCGAGTTCAAGCTCAAAATATTGGATGGATGGAT
TGGGCAAAAAATGGCGAAGCTGCTGGAACTTCTACGATGTCCTAC
CGTTTAGAAGCCATTCAAATTCAACTCGTTAAAAAAGGAAATCCA
GCTCCTGGAGCTACAACTTTTACTTTTTTGACTACTCCTTCCCTC
CAGTATTGCACTCAGGTTCAAAACATTGGCTGGCAAAATCCTGTA
TCTACTGGTCAGATCTCTGGAACGGTGGGTAAAAGCTTACGAACG
GAAGCTTTAAAAATTAATATTGGCAACCTTTCAGCAGGTATTGAC
GGTGGGGTATCGTATAGCAGCCAAATTCAAAATATTGGCTGGCAA
GCACCAGTATCCAATGGTCAAATCTCTGGAACTGTAGGACAAAGT
CTACGTTTAGAGGCCTTGAAAATAAACCTCACTGGTAGAATCTCA
CAGTATTTTGATATCAATTATGCCAGCCAAGTCCAAAACATCGGC
TGGCAAGCACCAGTATCCAACGGTCAAATCTCTGGAACTGTGGGA
ATGTCCTTACGAGTTGAAGCTGTAAAACTCTCTCTTTCTCCAAAA
TAA

In one aspect the autolysin is a homologue of SEQ ID NO: 1.

In one aspect, the homologue comprises a YjdB domain.

In one aspect the autolysin has at least 80% sequence identity to SEQ ID NO: 1 and comprises a YjdB domain.

In one aspect the autolysin is encoded by a polynucleotide sequence as set forth in SEQ ID NO: 1, or a sequence which has at least 80% sequence identity (such as at least 85%, at least 90%, 95%, 97%, 98%, or 99% identity) thereto. In one aspect the autolysin is encoded by a polynucleotide sequence as set forth in SEQ ID NO: 1, or a sequence which has at least 80% sequence identity (such as at least 85%, at least 90%, 95%, 97%, 98%, or 99% identity) thereto and comprises a YjdB domain. In one aspect, the autolysin additionally comprises a GH25 muramidase superfamily domain. In one aspect the autolysin is encoded by a polynucleotide sequence as set forth in SEQ ID NO: 1.

In one aspect, the bacterial strain is modified to reduce the activity and/or expression of an autolysin which corresponds to (or is equivalent to) the autolysin having the amino acid sequence SEQ ID NO: 2 of strain SL12699.

The amino acid sequence of the SL12699 autolysin is set forth in SEQ ID NO: 2:

(SEQ ID NO: 2)
MKLKKTHIISLILFSGLLFTKPVLAEDTISSDNTPMGHYIEQIQT
QETSKDSSSDTSSTTPNIRARSFAAAPTANVPSDLKSDDNTLPRK
DAVDIASYQSWMTQADFNSLKTSGVKSIVVKLTEGTNYTNPYAAN
QIKMAQNAGLNVAVYHYARLTGANSQSDANSLAIQEAAYFAKVAK
SFGLSSNIVMIMDCEQPYRDGSGNIIGPNPITVDWATAGVQFANT
LKANGYSNTKFYTSASWIGTDTATCQMNYNTLGGLKNLWAAQYLY
GKPSSSNLQNTQYGAWQYTSQMYYQGTSNLKANAVDTSIDYSNYF
TSTSPVPPSTYTITFNTDGGTPIANQSVAAGSVVSQPAAPTKAGE
NFSGWYSDSSLTQAYNFAKVITSNTTLYAKWIPITSPSISYQAQV
QNIGWQKNSYNGETAGTTGLGLRMESLKISLINLSNGLTNSNSHI
QYQGYVQNIGWQNPVQDGTIAGTVGQGLRFEAIKMNLSGEIANQY
DLYYRVQAQNIGWMDWAKNGEAAGTSTMSYRLEAIQIQLVKKGNP
APGATTFTFLTTPSLQYCTQVQNIGWQNPVSTGQISGTVGKSLRT
EALKINIGNLSAGIDGGVSYSSQIQNIGWQAPVSNGQISGTVGQS
LRLEALKINLTGRISQYFDINYASQVQNIGWQAPVSNGQISGTVG
MSLRVEAVKLSLSPK

In one aspect, the autolysin is a homologue of SEQ ID NO: 2.

In one aspect the autolysin comprises an amino acid sequence as set forth in SEQ ID NO: 2, or a sequence which has at least 80% sequence identity (such as at least 85%, at least 90%, 95%, 97%, 98%, or 99% identity) thereto.

In one aspect, the homologue comprises a YjdB domain.

In one aspect the autolysin comprises an amino acid sequence as set forth in SEQ ID NO: 2, or a sequence which has at least 80% sequence identity (such as at least 85%, at least 90%, 95%, 97%, 98%, or 99% identity) thereto, and a YjdB domain.

As used herein “YjdB” domain refers to a conserved and uncharacterized domain with an immunoglobulin-like (Ig-like) domain. Proteins comprising a YjdB domain may be identified by comparison with the YjdB Superfamily; for example using the National Center for Biotechnology Information (NCBI) Conserved Protein Domain Family Database (Lu et al., Nucleic Acids Res. 2020 Jan. 8; 48(D1):D265-D268). The YjdB domain is a member of the cl35007 superfamily.

Suitably, the autolysin may comprise a YjdB domain from SEQ ID NO: 2.

In one aspect, the YjdB domain may comprise or consist of an amino acid sequence which corresponds to about amino acid 394 to about amino acid 670 of SEQ ID NO: 2, or a portion thereof such as at least 100 amino acids, at least 150 amino acids, at least 200 amino acids, at least 250 amino acids or at least 270 amino acids. In one aspect, the YjdB domain may comprise or consist of an amino acid sequence which corresponds to about amino acid 394 to about amino acid 670 of SEQ ID NO: 2, or a sequence having at least 80% (such as at least 85%, at least 90%, at least 95%, at least 97%) identity thereto. In one aspect, the YjdB domain may comprise or consist of an amino acid sequence which corresponds to amino acid 394 to amino acid 670 of SEQ ID NO: 2, or a sequence having at least 80% (such as at least 85%, at least 90%, at least 95%, at least 97%) identity thereto.

In one aspect, the autolysin comprises a GH25 muramidase superfamily domain.

As used herein, “GH25 muramidase domain” refers to the glycosyl hydrolase 25 domain. The GH25 family comprises a group of hydrolases that act on the sugar moiety of the bacterial peptidoglycan (PG), cleaving the β-1-4 glycosidic bond between the MurNAc (N-acetyl muramic acid) and GlcNAc (N-acetyl-D-glucosamine) residues of the glycan strands. Proteins comprising a GH25 domain may be identified by comparison with GH25 domains in The Carbohydrate-Active Enzymes database https://www.cazypedia.org/ which provides classification of enzymes that assemble, modify and breakdown oligo and polysaccharides (Lombard et al., Nucleic Acids Research, Volume 42, Issue D1, 1 Jan. 2014, Pages D490-D495).

In one aspect, the GH25 domain may comprise or consist of an amino acid sequence which corresponds to about amino acid 90 to about amino acid 311 of SEQ ID NO: 2, or a portion thereof such as at least 100 amino acids, at least 150 amino acids, at least 200 amino acids, or at least 220 amino acids. In one aspect, the GH25 domain may comprise or consist of an amino acid sequence which corresponds to about amino acid 90 to about amino acid 311 of SEQ ID NO: 2, or a sequence having at least 80% (such as at least 85%, at least 90%, at least 95%, at least 97%) identity thereto. In one aspect, the GH25 domain may comprise or consist of an amino acid sequence which corresponds to amino acid 90 to amino acid 311 of SEQ ID NO: 2, or a sequence having at least 80% (such as at least 85%, at least 90%, at least 95%, at least 97%) identity thereto.

In one aspect, the bacterial strain according to the present invention has been modified to comprise an autolysin^{R} allele encoding an autolysin^{R} protein, wherein said autolysin^{R} allele reduces the sensitivity to phage D6867 of a Lactococcus lactis SL12699 derivative strain, said SL12699 derivative strain being a Lactococcus lactis SL12699 strain in which the autolysin allele has been replaced by said autolysin^{R} allele, and wherein sensitivity to phage D6867 is determined by Efficiency of Plaquing (EOP) Assay I as described herein.

The present inventors have surprisingly found that an autolysin^{R} allele, such that the gene encodes an autolysin^{R} protein, reduces the sensitivity of bacterial strains (such as lactic acid bacteria) to at least one phage, such as P335-like phage e.g. D6867.

In an aspect, the present invention provides a method to identify an autolysin^{R} allele encoding an autolysin^{R} protein, comprising:
a) introducing the autolysin^{R} allele to be tested (candidate autolysin^{R} allele) in lieu of the allele of the autolysin gene of Lactococcus lactis SL12699, to obtain a SL12699 derivative strain; and
b) determining by Efficiency of Plaquing Assay I the EOP of phage D6867 on the SL12699 derivative strain of step a), wherein an EOP reduction of at least 4 log is indicative of an autolysin^{R} allele which is a autolysin^{R} allele encoding an autolysin^{R} protein.

As used herein, the expression “an allele of the autolysin^{R} gene” means the version of the autolysin gene found in a particular bacterium. As for most of the bacterial genes, the nucleotide sequence of a gene can vary, and alleles represent the different sequences of the same gene. Thus, the allele of the autolysin gene of Lactococcus lactis SL12699 is as set forth in SEQ ID NO:1. This allele as defined in SEQ ID NO: 1 encodes an autolysin protein as set forth in SEQ ID NO:2.

The inventors have shown that certain autolysin alleles are able to reduce the sensitivity of Lactococcus lactis SL12699 to phage D6867 when they are inserted in lieu f the original (i.e. native) allele of the autolysin gene (SEQ ID NO:1) of SL12699. These alleles are defined herein as “autolysin^{R} alleles”. The protein encoded by these autolysin^{R} alleles is referred to herein as “autolysin^{R} protein.

Thus, any autolysin^{R} allele (encoding an autolysin^{R} protein) reducing the sensitivity of the SL12699 strain to phage D6867 (as defined herein) is part of the invention. In other words, an autolysin^{R} allele is defined as an autolysin^{R} allele which reduces the sensitivity to phage D6867 of a Lactococcus lactis SL12699 derivative strain, said SL12699 derivative strain being a Lactococcus lactis SL12699 into which its original autolysin allele has been replaced by said autolysin^{R} allele.

The term “reduces the sensitivity” may include “increases the resistance”, “improves the resistance”, “enhances the resistance”, “confers resistance” and “improves tolerance”.

In one aspect, the modification which reduces the activity and/or expression of autolysin may be a deletion. The deletion may result in deletion of all or part of the autolysin gene. In other words, the autolysin gene is knocked out. Suitably, the deletion may result in deletion of all or part of an autolysin gene which corresponds to (or is equivalent to) the autolysin having the amino acid sequence SEQ ID NO:2 in strain SL12699.

In one aspect, the deletion removes at least part of the YjdB domain. In one aspect, the deletion removes all of the YjdB domain. In one aspect, the deletion is a single nucleotide deletion. In one aspect, the deletion is a deletion of at least 10 nucleotides, at least 20, at least 30, at least 40, at least 50, at least 60, at least 70, at least 80, at least 90, at least 100, at least 110, at least 120, at least 130, at least 140, at least 150, at least 160, at least 170, at least 180, at least 200 nucleotides. In one aspect, the deletion of at least part of the YjdB domain described herein, results in reduced activity and/or expression of autolysin and increased resistance to bacteriophage. In one aspect, the deletion of at least part of the YjdB domain described herein, results in reduced activity and/or expression of autolysin and increased resistance to bacteriophage.

In a preferred aspect, the deletion corresponds to about a 153 nucleotide deletion starting at position 1,225 of the autolysin gene encoded by SEQ ID NO: 1.

In one aspect, the modification which reduces the activity and/or expression of autolysin may be a mutation which introduces an early stop codon. Suitably, the early stop codon may result in the expression of a truncated autolysin protein, which is an autolysin^{R} protein. For example, the early stop codon may result in a truncation of at least, 10, at least 20, at least 30, at least 40, at least 50, at least 60, at least 70, at least 80, at least 90, at least 100, at least 150, at least 200, at least 250, at least 300, at least 400, at least 450, at least 500, or more amino acids from the C terminal of the protein. In a preferred aspect, the truncation results in deletion of at least part of, or all of the YjdB domain.

In a preferred aspect, the modification corresponds to an early stop codon at about amino acid position 628 of the autolysin encoded by SEQ ID NO: 2.

In a preferred aspect, the modification corresponds to an early stop codon at about amino acid position 410 of the autolysin encoded by SEQ ID NO: 2.

### Bacterial Strain

In one aspect, the bacterial strain according to the present invention is a lactic acid bacterial strain.

As used herein the term “lactic acid bacteria” or “lactic acid bacterium” or “LAB” refers to Gram positive bacteria which ferment sugars to produce exclusively or predominantly lactic acid.

The industrially most useful lactic acid bacteria are found among the genera Lactococcus, Streptococcus, Lactobacillus, Leuconostoc, Pediococcus, Enterococcus, Oenococcus and Bifidobacterium. In one embodiment, it is therefore preferred that the lactic acid bacterium is selected from this group of genera.

In one aspect, the bacterial strain belongs to the Lactococcus genus.

Lactococcus subsp. are amongst the lactic acid bacteria most affected by bacteriophage infection.

In one aspect, the bacterial strain is a Lactococcus sp., suitably, the strain may be from a Lactococcus lactis species.

Suitably, the bacterial strain may be selected from Lactococcus lactis subsp. cremoris, Lactococcus lactis subsp, lactis or Lactococcus lactis subsp. lactis biovar. diacetylactis.

In some preferred embodiments, the bacterial strain of the invention has reduced sensitivity or is resistant to at least one phage. In one embodiment, the at least one phage is a P335-like phage. In a preferred embodiment, the at least one phage is D6867.

One way to measure the bacteriophage sensitivity of a bacterium is in a standard EOP assay using a suitable bacteriophage or panel of bacteriophages. Accordingly, in one embodiment the bacteriophage sensitivity (or resistance) of a bacterial strain of the invention is determined in a standard EOP assay. In a preferred embodiment, the bacteriophage sensitivity (or resistance) of a lactic acid bacterium of the invention is determined in the Efficiency of Plaquing Assay I described herein.

In one embodiment, the bacterial strain of the invention has reduced sensitivity to at least one phage (such as a P335-like phage) relative to the corresponding bacterial strain which has not been modified to reduce the activity and/or expression of autolysin.

In one aspect, the bacterial strain comprises an autolysin gene which encodes an autolysin comprising a YjdB domain. In one aspect, the bacterial strain comprises an autolysin gene which encodes an autolysin comprising a YjdB domain and a GH25 domain.

In one aspect, the bacterial strain prior to modification comprises an autolysin gene which encodes an autolysin comprising a YjdB domain or both a YjdB domain and a GH25 domain.

In some aspects, the parental strain comprises an autolysin gene which encodes an autolysin comprising a YjdB domain or both a YjdB domain and a GH25 domain.

In one aspect, the bacterial strain according to the present invention maintains commercially acceptable milk acidification rates. In other words, the modification to reduce the activity and/or expression of autolysin does not significantly impair the milk acidification rate of the bacterial strain.

Methods for measuring milk acidification rates are known in the art. For example in WO2006042862. Milk acidification rates may be measured as described in the Examples herein.

A milk acidification assay may comprise:
growing a strain overnight in 11% non fat dairy milk (NFDM);
transferring growth into M17-Lac;
once the cultures reach an OD₆₀₀ of approximately 0.65, inoculating at 0.75% into activity milk (commercial 1% fat Kemp's);
keeping the cultures in a 30° C. water bath overnight with pH probes and monitoring acidification of each strain about every 2 minutes.

### Bacteriophage

As used herein, the term “bacteriophage” has its conventional meaning as understood in the art, i.e. a virus that infects bacteria. Many bacteriophages are specific to a particular genus or species or strain of bacteria. The term “bacteriophage” is synonymous with the term “phage”.

The phages of three species, P335, 936 and c2 are mainly responsible for milk fermentation failures worldwide.

In one aspect, the bacterial strain according to the present invention has reduced sensitivity to a bacteriophage selected from a P335-type phage, a P335-like phage, 936-type phage, a 936-like phage, a c2-type phage or a c2-like phage.

Phages which are described as “like” refer to phages which share structural and/or functional or mechanistic similarity with members of a certain family.

Phage P335 belongs to the Siphoviridae family and is a virulent type phage for the Lactococcus lactis species. P335 phages have previously been classified into four sub-groups (I-IV). The family of P335 phages include the reference phage P335, Tuc2009, RP901-1, BK5-T, rlt, LC3 ul36 and 4268. These phages were given this group assignment based on Southern hybridization and morphological analyses with subsequent confirmation by comparative sequence analysis. L. lactis genomic projects have also demonstrated the presence of prophages and prophage remnants in some lactococcal strains.

In one aspect, the bacteriophage is a P335-like bacteriophage.

As used herein “P335-like” phage refers to a phage which shares structural and/or functional similarity with members of the P335 family.

P335-type phage that infect species of bacteria including lactic acid bacteria e.g. strains of the Lactococcal genus are known and can be identified based on their function or on genetic or genomic analyses.

The sequences of numerous phages including P335 type phages are known and are available in public databases for example the GenBank accession numbers for several P335 phages are as follows: Tuc2009 (NC_002703.1); TP901-1 (NC_002747.1); LC3 (NC_005822.1); P335 (Q838728.1); ul36 (NC_004066.1); Q33 (JX564242.1); BK5-T (NC_002796.1); r1t (NC_004302.1) and BM13 (NC_021861.1).

In one aspect, a P335-like phage has at least 80%, at least 85%, at least 90%, at least 95%, at least 97%, at least 98%, at least 99% sequence identity with the nucleotide sequence of a P335 phage described above. In one aspect, a P335-like phage has at least 80%, at least 85%, at least 90%, at least 95%, at least 97%, at least 98%, at least 99% sequence identity with the amino acid sequence of a P335 phage described above.

Mahony et al., (Mahony et al. BMC Genomics (2017) 18:146) have also described multiplex PCR methods for the classification of P335 phages. Seven pairs of primers were designed to facilitate the detection and classification of emerging P335 phage isolates, based on the RBP-encoding genes of phages belonging to sub-groups and receptor binding protein (RBP)-related sequences.

An example of a P335-like phage which infects bacteria, such as LAB bacteria such as bacteria of the Lactococcus genus, is the phage D6867. The D6867 phage was is available at the Leibniz Institute DSMZ-German Collection of Microorganisms and Cell Cultures under Accession number: DSM 33596). The host strain of this phage is Lactococcus lactis ssp. lactis SL12699 which is available at the Leibniz Institute DSMZ-German Collection of Microorganisms and Cell under Accession number: DSM 3360.

In one aspect, a P335-like phage has at least 80%, at least 85%, at least 90%, at least 95%, at least 97%, at least 98%, at least 99% sequence identity with the nucleotide sequence of D6867. In one aspect, a P335-like phage has at least 80%, at least 85%, at least 90%, at least 95%, at least 97%, at least 98%, at least 99% sequence identity with the amino acid sequence of D6867.

In a preferred aspect, the bacteriophage is D6867.

Accordingly, this preferred phage can be used to determine the resistance (or sensitivity) to bacteriophage conferred by a modification (such as mutation, or autolysin^{R}) of the invention, such as in the Efficiency of Plaquing Assay I described herein.

Another example of a P335-like phage which infects bacteria, such as LAB bacteria such as bacteria of the Lactococcus genus, is the phage D7138.

D6867 and D7138 have high similarity to a prophage in SL12699 (85.1% and 95.8% pairwise identity respectively).

In one aspect, the bacteriophage has at least 80% sequence identity to a P335-like or a P335 prophage in the bacterial strain. In other words, the bacteriophage is a virulent prophage which has previously excised itself from the bacterial strain.

### Polynucleotide

In one aspect, the present invention provides a polynucleotide sequence which reduces the activity and/or expression of autolysin in a bacterial strain.

In one aspect, the present invention provides a polynucleotide comprising an autolysin^{R} allele of the invention. In one aspect, the polynucleotide is an autolysin^{R} allele of the invention. In another embodiment, the polynucleotide encodes an autolysin^{R} protein of the invention.

The term “polynucleotide” in relation to the present invention includes genomic DNA, cDNA, and synthetic DNA. Preferably it means DNA, more preferably cDNA.

In one embodiment the term “polynucleotide” means cDNA.

Typically, the polynucleotide encompassed by the scope of the present invention is recombinant and is prepared using recombinant DNA techniques (i.e. recombinant DNA), as described herein. However, in an alternative embodiment of the invention, the polynucleotide could be synthesised, in whole or in part, using chemical methods well known in the art (see Caruthers M H et al., (1980) Nuc Acids Res Symp Ser 215-23 and Horn T et al., (1980) Nuc Acids Res Symp Ser 225-232).

A polynucleotide according to the present invention which has the specific properties as defined herein or a protein which is suitable for modification may be identified and/or isolated and/or purified from any cell or organism producing said protein. Various methods are well known within the art for the identification and/or isolation and/or purification of polynucleotides.

By way of example, PCR amplification techniques to prepare more of a polynucleotide may be used once a suitable polynucleotide has been identified and/or isolated and/or purified. By way of further example, a genomic DNA and/or cDNA library may be constructed using chromosomal DNA or messenger RNA from the organism producing the protein. If the amino acid sequence of the protein is known, labelled oligonucleotide probes may be synthesised and used to identify protein-encoding clones from the genomic library prepared from the organism. Alternatively, a labelled oligonucleotide probe containing sequences homologous to another known protein gene could be used to identify protein-encoding clones. In the latter case, hybridisation and washing conditions of lower stringency are used.

Alternatively, the polynucleotide according to the present invention may be a synthetic polynucleotide, prepared synthetically by established standard methods, e.g. the phosphoroamidite method described by Beucage S. L. et al., 1981, Tetrahedron Letters 22:1859-1869, or the method described by Matthes et al., 1984, EMBO J., 3:801-805. In the phosphoroamidite method, oligonucleotides are synthesised, e.g. in an automatic DNA synthesiser, purified, annealed, ligated and cloned in appropriate vectors.

The polynucleotide may be of mixed genomic and synthetic origin, mixed synthetic and cDNA origin, or mixed genomic and cDNA origin, prepared by ligating fragments of synthetic, genomic or cDNA origin (as appropriate) in accordance with standard techniques. Each ligated fragment corresponds to various parts of the entire polynucleotide. The polynucleotide may also be prepared by polymerase chain reaction (PCR) using specific primers, for instance as described in U.S. Pat. No. 4,683,202 or in Saiki R K et al., 1988, Science, 239:487-491.

The polynucleotide encompassed by the present invention may be isolated or substantially purified. By “isolated” or “substantially purified” is intended that the polynucleotides are substantially or essentially free from components normally found in association with the polynucleotide in its natural state. Such components include other cellular material, culture media from recombinant production, and various chemicals used in chemically synthesising the nucleic acids.

An “isolated” polynucleotide or nucleic acid is typically free of nucleic acid sequences that flank the nucleic acid of interest in the genomic DNA of the organism from which the nucleic acid was derived (such as coding sequences present at the 5′ or 3′ ends). However, the molecule may include some additional bases or moieties that do not deleteriously affect the basic characteristics of the composition.

### Vectors and Constructs

The polynucleotide sequence(s) described herein may be present in a vector. The polynucleotide sequence may be operably linked to regulatory sequences such that the regulatory sequences are capable of providing for the expression of the polynucleotide sequence by a suitable host organism, i.e. the vector may be an expression vector.

The term “expression vector” means a construct capable of in vivo or in vitro expression. Preferably, the expression vector is incorporated in the genome of the organism. The term “incorporated” preferably covers stable incorporation into the genome.

The vectors may be transformed into a suitable bacterial host cell to provide for expression of a polypeptide having the specific properties as defined herein.

The choice of vector, e.g. plasmid, cosmid, virus or phage vector, will often depend on the host cell into which it is to be introduced.

The vectors may contain one or more selectable marker genes—such as a gene which confers antibiotic resistance e.g. ampicillin, kanamycin, chloramphenicol or tetracyclin resistance. Alternatively, the selection may be accomplished by co-transformation (as described in WO91/17243).

The vector may further comprise a nucleotide sequence enabling the vector to replicate in the host cell in question. Examples of such sequences are the origins of replication of plasmids pUC19, pACYC177, pUBI 10, pE194, pAMBI and pIJ702.

The term “construct”-which is synonymous with terms such as “cassette”-includes a polynucleotide sequence, directly or indirectly attached to a promoter.

An example of an indirect attachment is the provision of a suitable spacer group such as an intron sequence, such as the Shl-intron or the ADH intron, intermediate the promoter and the nucleotide sequence of the present invention. In some cases, the terms do not cover the natural combination of the polynucleotide sequence coding for the protein ordinarily associated with the wild type gene promoter and when they are both in their natural environment.

The construct may contain or express a marker, which allows for the selection of the genetic construct.

In one aspect, the present invention provides a vector or construct comprising a polynucleotide sequence according to the present invention. In one aspect, the present invention provides a vector or construct comprising a polynucleotide sequence which reduces the activity and/or expression of autolysin in a bacterial strain. In one aspect, the present invention provides a vector or construct comprising an autolysin^{R} allele of the invention. In another embodiment, the vector or construct comprises a polynucleotide which encodes an autolysin^{R} protein of the invention.

In one aspect, the invention is directed to use of a polynucleotide, construct or vector of the invention to reduce the sensitivity of a bacterial strain to at least one bacteriophage (such as a P335-like phage).

### Bacterial Composition

The invention is also directed to a bacterial composition comprising or consisting of at least one bacterial strain of the invention. In one aspect, the bacterial strain is a lactic acid bacterial strain. In one embodiment, the bacterial composition is a pure culture, i.e., comprises or consists of a single bacterium strain. In another embodiment, the bacterial composition is a mixed culture, i.e. comprises or consists of at least one bacterial strain(s) of the invention and at least one other bacterial strain. By “at least one other bacterium strain”, it is meant 1 or more, and in particular 1, 2, 3, 4 or 5 strains.

In one aspect, the bacterial strain according to the present invention is from any of the following genera Lactococcus, Lactobacillus, Leuconostoc, Pediococcus, Streptococcus, Aerococcus, Carnobacterium, Enterococcus, Oenococcus, Sporolactobacillus, Tetragenococcus, Vagococcus, Weissella and Bifidobacterium.

In one embodiment, a bacterial composition of the invention comprises or consists of at least one bacterial strain(s) of the invention, and one or more further bacterium of the species selected from the group consisting of Lactococcus, Lactobacillus, Leuconostoc, Pediococcus, Streptococcus, Aerococcus, Carnobacterium, Enterococcus, Oenococcus, Sporolactobacillus, Tetragenococcus, Vagococcus, Weissella and Bifidobacterium.

In one embodiment, a bacterial composition of the invention comprises or consists of at least one bacterial strain(s) of the invention, and one or more further bacterium of the species selected from the group consisting of Lactococcus species, a Streptococcus species, a Lactobacillus species including Lactobacillus acidophilus, an Enterococcus species, a Pediococcus species, a Leuconostoc species, a Bifidobacterium species and an Oenococcus species or any combination thereof.

In one embodiment, a bacterial composition of the invention comprises or consists of at least one bacterial strain(s) of the invention, and one or more further bacterium of the Streptococcus species.

Lactococcus species include Lactobacillus acidophilus and Lactococcus lactis, including Lactococcus lactis subsp. lactis, Lactococcus lactis subsp. cremoris and Lactococcus lactis subsp. lactis biovar diacetylactis. Bifidobacterium species includes Bifidobacterium animalis, in particular Bifidobacterium animalis subsp lactis. Other lactic acid bacteria species include Leuconostoc sp., Streptococcus thermophilus, Lactobacillus delbrueckii subsp. bulgaricus, and Lactobacillus helveticus.

In a particular aspect of any bacterial composition defined herein, either as a pure or mixed culture, the bacterial composition further comprises at least one probiotic strain such as Bifidobacterium animalis subsp. lactis, Lactobacillus acidophilus, Lactobacillus paracasei, or Lactobacillus casei.

In a particular aspect, the bacterial composition, either as a pure or mixed culture as defined above is in frozen, dried, freeze-dried, liquid or solid format, in the form of pellets or frozen pellets, or in a powder or dried powder. In a particular aspect, the bacterial composition of the invention is in a frozen format or in the form of pellets or frozen pellets, in particular contained into one or more box or sachet. In another aspect, the bacterial composition as defined herein is in a powder form, such as a dried or freeze-dried powder, in particular contained into one or more box or sachet.

In a particular aspect, the bacterial composition of the invention, either as a pure culture or mixed culture as defined above, and whatever the format (frozen, dried, freeze-dried, liquid or solid format, in the form of pellets or frozen pellets, or in a powder or dried powder) comprises the bacterial strain(s) of the invention in a concentration comprised in the range of 10⁵ to 10¹² cfu (colony forming units) per gram of the bacterial composition. In a particular aspect, the concentration of the bacterial strain(s) within the bacterial composition of the invention is in the range of 10⁷ to 10¹² cfu per gram of the bacterial composition, and in particular at least 10⁷, at least 10⁸, at least 10⁹, at least 10¹⁰ or at least 10¹¹ CFU/g of the bacterial composition. In a particular aspect, when in the form of frozen or dried concentrate, the concentration of bacterial strain(s) of the invention—as pure culture or as a mixed culture-within the bacterial composition is in the range of 10⁸ to 10¹² cfu/g of frozen concentrate or dried concentrate, and more preferably at least 10⁸, at least 10⁹, at least 10¹⁰, at least 10¹¹ or at least 10¹² cfu/g of frozen concentrate or dried concentrate.

In one aspect, the bacterial compositions according to the invention is a starter culture. Starter cultures used in the manufacture of many fermented milk, cheese and butter products include cultures of bacteria, generally classified as lactic acid bacteria. Such bacterial starter cultures impart specific features to various dairy products by performing a number of functions.

Commercial non-concentrated cultures of bacteria are referred to in industry as ‘mother cultures’, and are propagated at the production site, for example a dairy, before being added to an edible starting material, such as milk, for fermentation.

The starter culture may comprise several bacterial strains, i.e. it may be a defined mixed culture. Accordingly, the starter culture may comprise the bacterial strain of the invention and a further bacterial strain.

For example, the starter culture may be suitable for use in the dairy industry. When used in the dairy industry the starter culture may additionally comprise a lactic acid bacteria species, a Bifidobacterium species, a Brevibacterium species, and/or a Propionibacterium species. Cultures of lactic acid bacteria are commonly used in the manufacture of fermented milk products—such as buttermilk, yoghurt or sour cream, and in the manufacture of butter and cheese, for example Brie or Harvati.

Suitable lactic acid bacteria include commonly used strains of a Lactococcus species, a Streptococcus species, a Lactobacillus species including Lactobacillus acidophilus, Enterococcus species, Pediococcus species, a Leuconostoc species and Oenococcus species or combinations thereof.

Lactococcus species include the widely used Lactococcus lactis, including Lactococcus lactis subsp. lactis, Lactococcus lactis subsp. lactis biovar diacetylactis and Lactococcus lactis subsp. cremoris.

Other lactic acid bacteria species include Leuconostoc sp., Streptococcus thermophilus, Lactobacillus delbrueckii subsp. bulgaricus and Lactobacillus helveticus. Mesophilic cultures of lactic acid bacteria commonly used in the manufacture of fermented milk products such as buttermilk, yoghurt or sour cream, and in the manufacture of butter and cheese, for example Brie or Harvati. In addition, probiotic strains such as Bifidobacterium lactis, Lactobacillus acidophilus, Lactobacillus casei may be added during said manufacturing to enhance flavour or to promote health.

Cultures of lactic acid bacteria commonly used in the manufacture of cheddar and Monterey Jack cheeses include Streptococcus thermophilus, Lactococcus lactis subsp. lactis and Lactococcus lactis subsp. cremoris or combinations thereof.

Thermophilic cultures of lactic acid bacteria commonly used in the manufacture of Italian cheeses such as Pasta filata or parmesan, include Streptococcus thermophilus and Lactobacillus delbrueckii subsp. bulgaricus. Other Lactobacillus species—such as Lactobacillus helveticus—may be added during manufacturing to obtain a desired flavour. The selection of organisms for the starter culture of the invention will depend on the particular type of products to be prepared and treated. Thus, for example, for cheese and butter manufacturing, mesophillic cultures of Lactococcus species, Leuconostoc species and Lactobacillus species are widely used, whereas for yoghurt and other fermented milk products, thermophilic strains of Streptococcus species and of Lactobacillus species are typically used.

Starter cultures may be prepared by techniques well known in the art such as those disclosed in U.S. Pat. No. 4,621,058. By way of example, starter cultures may be prepared by the introduction of an inoculum, for example a bacterium, to a growth medium to produce an inoculated medium and ripening the inoculated medium to produce a starter culture.

Dried starter cultures may be prepared by techniques well known in the art, such as those discussed in U.S. Pat. Nos. 4,423,079 and 4,140,800.

### Product

Any product, which is prepared from, contains or comprises a bacterium or bacterial composition of the invention is contemplated in accordance with the present invention.

Suitable products include, but are not limited to a food or a feed product.

These include, but are not limited to, fruits, legumes, fodder crops and vegetables including derived products, grain and grain-derived products, dairy foods and dairy food-derived products, meat, poultry and seafood. Preferably, the food or feed product is a dairy, meat or cereal product.

The term “food” is used in a broad sense and includes feeds, foodstuffs, food ingredients, food supplements, and functional foods. Here, the term “food” is used in a broad sense—and covers food for humans as well as food for animals (i.e. a feed). In a preferred aspect, the food is for human consumption.

As used herein the term “food ingredient” includes a formulation, which is or can be added to foods and includes formulations which can be used at low levels in a wide variety of products that require, for example, acidifying or emulsifying.

As used herein, the term “functional food” means a food which is capable of providing not only a nutritional effect and/or a taste satisfaction, but is also capable of delivering a further beneficial effect to consumer. Although there is no legal definition of a functional food, most of the parties with an interest in this area agree that there are foods marketed as having specific health effects.

The bacterial strain or bacterial composition of the present invention may be—or may be added to—a food ingredient, a food supplement, or a functional food.

The food may be in the form of a solution or as a solid-depending on the use and/or the mode of application and/or the mode of administration.

The bacterial strain or bacterial composition of the present invention can be used in the preparation of food products such as one or more of: confectionery products, dairy products, meat products, poultry products, fish products and bakery products.

By way of example, the bacterial strain or bacterial composition can be used as an ingredient to prepare soft drinks, a fruit juice or a beverage comprising whey protein, health teas, cocoa drinks, milk drinks and lactic acid bacteria drinks, yoghurt, drinking yoghurt and wine.

Preferably a food as described herein is a dairy product. More preferably, a dairy product as described herein is one or more of the following: a yoghurt, a cheese (such as an acid curd cheese, a hard cheese, a semi-hard cheese, a cottage cheese), a buttermilk, quark, a sour cream, kefir, a fermented whey-based beverage, a koumiss, a milk beverage, a yoghurt drink, a fermented milk, a matured cream, a cheese, a fromage frais, a milk, a dairy product retentate, a process cheese, a cream dessert, or infant milk.

Preferably, a food as described herein is a fermented food product. More preferably, a food as described herein is a fermented dairy product—such as a fermented milk, a yoghurt, a cream, a matured cream, a cheese, a fromage frais, a milk beverage, a processed cheese, a cream dessert, a cottage cheese, a yoghurt drink, a dairy product retentate, or infant milk. Preferably the dairy product according to the invention comprises milk of animal and/or plant origin.

Milk is understood to mean that of animal origin, such as cow, goat, sheep, buffalo, zebra, horse, donkey, or camel, and the like. The term milk also applies to what is commonly called vegetable milk, that is to say extracts of plant material which have been treated or otherwise, such as leguminous plants (soya bean, chick pea, lentil and the like) or oilseeds (colza, soya bean, sesame, cotton and the like), which extract contains proteins in solution or in colloidal suspension, which are coagulable by chemical action, by acid fermentation and/or by heat. The word milk also denotes mixtures of animal milks and of vegetable milks.

The milk may be in the native state, reconstituted milk, a skimmed milk or a milk supplemented with compounds necessary for the growth of the bacteria or for the subsequent processing of fermented milk, such as fat, proteins of a yeast extract, peptone and/or a surfactant, for example.

In one embodiment, the term “milk” means commercial UHT milk supplemented with 3% (w/w) of semi-skimmed milk powder pasteurized by heating during 10 min+/−1 min. at 90° C.+/−0.2° c.

In a further aspect there is provided a method for manufacturing a fermented product comprising a) inoculating a substrate with the bacterial strain according to the present invention or the bacterial composition according to the invention and b) fermenting the inoculated substrate to obtain a fermented product. In a particular embodiment, the bacterial strain(s) of the invention is inoculated as a bacterial composition according to the present invention, such as a pure culture or a mixed culture. Preferably, the substrate is a milk substrate, more preferably milk. By “milk substrate”, it is meant milk of animal and/or plant origin. In a particular embodiment, the milk substrate is of animal origin, such as cow, goat, sheep, buffalo, zebra, horse, donkey, or camel, and the like. The milk may be in the native state, a reconstituted milk, a skimmed milk, or a milk supplemented with compounds necessary for the growth of the bacteria or for the subsequent processing of fermented milk. Preferably, the milk substrate comprises solid items. Preferably, the solid items comprise or consist of fruits, chocolate products, or cereals. Preferably, the fermented product is a fermented dairy product.

The present invention also provides in a further aspect the use of the bacterial strain or bacterial composition according to the present invention to manufacture a food or feed product, preferably a fermented food product, more preferably a fermented dairy product. The invention is also directed to a fermented dairy product, which is obtained using the bacterial strain(s) or bacterial composition of the invention, in particular obtained or obtainable by the method of the invention. Thus, the invention is directed to a fermented dairy product comprising the bacterial strain(s) or bacterial composition of the invention.

In a particular embodiment, the fermented dairy food product of the invention is fresh fermented milk.

Preparation of a Bacterial Strain with Reduced Sensitivity to at Least One Phage

In one aspect, the present invention provides a method for increasing (e.g. conferring or increasing) the resistance of a bacterial strain to bacteriophage (such as at least one P335-like phage) comprising modifying said bacterial strain by decreasing the activity and or expression of autolysin.

Suitably, said bacterial strain may maintain commercially acceptable milk acidification kinetics. Suitably, said strain is for use in food/feed production in particular, the production of fermented products such as fermented dairy products.

In one aspect, the present invention provides a method for preparing at least one bacteriophage resistant variant strain (such as resistance to at least one P335-like phage), comprising modifying a parental bacterial strain to decrease the activity and or expression of autolysin.

Suitably, said bacterial strain may maintain commercially acceptable milk acidification kinetics. Suitably, said strain is for use in food/feed production in particular, the production of fermented products such as fermented dairy products.

In one aspect, a method according to the present invention comprises:
a) providing a bacterial strain (such as a strain of the Lactococcus genus) sensitive to at least one bacteriophage (such as a P335-like phage);
b) modifying said bacterial strain to reduce the activity and/or expression of autolysin; and
c) recovering the strain(s) having reduced sensitivity to at least one bacteriophage (such as a P335-like phage), optionally wherein sensitivity to at least one bacteriophage (such as a P335-like phage) is determined by EOP Assay I.

By way of example, the present method may comprise:
providing a mutation in a nucleic acid sequence which encodes an autolysin protein, such as a protein comprising the amino acid sequence shown as SEQ ID NO: 2, or an amino acid sequence which has at least 80% sequence identity thereto (preferably at least 85%, preferably at least 90%, preferably at least 96%, preferably at least 98% sequence identity thereto) or a homologue of SEQ ID NO:2;
providing a mutation in a promoter of a nucleic acid sequence which encodes an autolysin protein such as a protein comprising the amino acid sequence shown as SEQ ID NO: 2 or an amino acid sequence which has at least 80% sequence identity thereto (preferably at least 85%, preferably at least 90%, preferably at least 96%, preferably at least 98% sequence identity thereto) or a homologue of SEQ ID NO:2;
providing a mutation in a nucleic acid sequence of an autolysin gene, such as a gene which comprises SEQ ID NO:1, or a nucleotide sequence which has at least 80% sequence identity thereto (preferably at least 85%, preferably at least 90%, preferably at least 96%, preferably at least 98% sequence identity thereto) or a homologue of SEQ ID NO:1;
providing a mutation in a promoter of a nucleic acid sequence of an autolysin gene, such as a gene which comprises SEQ ID NO:1, or a nucleotide sequence which has at least 80% sequence identity thereto (preferably at least 85%, preferably at least 90%, preferably at least 96%, preferably at least 98% sequence identity thereto) or a homologue of SEQ ID NO: 1;
providing an antisense RNA, siRNA or miRNA which reduces the level of nucleic acid sequence encoding an autolysin protein, such as a protein comprising the amino acid sequence shown as SEQ ID NO:2 or an amino acid sequence which has at least 80% sequence identity thereto (preferably at least 85%, preferably at least 90%, preferably at least 96%, preferably at least 98% sequence identity thereto) or a homologue of SEQ ID NO:2;
providing an antisense RNA, siRNA or miRNA which reduces the level of an autolysin nucleic acid sequence, such as an autolysin gene comprising the nucleotide sequence shown as SEQ ID NO:1 or a nucleotide sequence which has at least 80% sequence identity thereto (preferably at least 85%, preferably at least 90%, preferably at least 96%, preferably at least 98% sequence identity thereto) or a homologue of SEQ ID NO: 1.

In one embodiment, the bacterial strain according to the present invention has been modified to reduce the activity and/or expression of autolysin. In one aspect, the modification may render autolysin partially or completely non-functional with respect to phage infection.

In one aspect, the modification is a stop codon, an insertion, a deletion or a mutation. In other words, the bacterial strain has been modified to introduce a stop codon, an insertion, a deletion or a mutation which reduces the activity and/or expression of autolysin.

In one aspect, the modification has been introduced into a nucleic acid sequence which encodes said autolysin, or in to a regulatory region (such as a promoter, operon or enhancer) which contributes to controlling the expression of said autolysin. In other words, a modification (such as a mutation, a stop codon, an insertion, or a deletion) which reduces the activity and/or expression of autolysin has been introduced to the coding region or into a regulatory region.

In a further aspect, the invention is directed to a bacterial obtainable or obtained by a method of the invention.

### Sequence Identity

In addition to the specific amino acid sequences and polynucleotides mentioned herein, the present invention encompasses variants, homologues, derivatives and fragments thereof.

The term “variant” is used to mean a naturally occurring nucleotide sequence or amino acid sequence which differs from a wild-type sequence.

The term “homologue” means an entity having a certain homology with the subject nucleotide sequences. Here, the term “homology” can be equated with “identity”.

In the present context, a homologous sequence is taken to include an amino acid or a nucleotide sequence which may be at least 80, 85 or 90% identical, preferably at least 95%, 96%, 97%, 98% or 99% identical to the subject sequence. Typically, the homologues will comprise the same active sites etc. as the subject amino acid sequence for instance. Although homology can also be considered in terms of similarity (i.e. amino acid residues having similar chemical properties/functions), in the context of the present invention it is preferred to express homology in terms of sequence identity.

In one aspect, a homologous sequence of an autolysin gene may comprise a nucleotide sequence which is at least 80% identical to SEQ ID NO:1 and a YjdB domain. In one aspect, a homologous sequence of an autolysin gene may comprise a nucleotide sequence which is at least 80% identical to SEQ ID NO:1, a YjdB domain and a GH25 domain.

In one aspect, a homologous sequence of an autolysin protein may comprise an amino acid sequence which is at least 80% identical to SEQ ID NO:2 and a YjdB domain. In one aspect, a homologous sequence of an autolysin protein may comprise an amino acid sequence which is at least 80% identical to SEQ ID NO:2, a YjdB domain and a GH25 domain.

In one aspect, a homologous sequence is taken to include an amino acid sequence or nucleotide sequence which has one or several additions, deletions and/or substitutions compared with the subject sequence.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible-reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons.

Calculation of maximum % homology or % identity therefore firstly requires the production of an optimal alignment, taking into consideration gap penalties. A suitable computer program for carrying out such an alignment is the Vector NTI (Invitrogen Corp.). Examples of software that can perform sequence comparisons include, but are not limited to, the BLAST package (see Ausubel et al., 1999, Short Protocols in Molecular Biology, 4th Ed—Chapter 18), BLAST 2 (see FEMS Microbiol Lett, 1999, 174(2): 247-50; FEMS Microbiol Lett, 1999, 177(1): 187-8), FASTA (Altschul et al., 1990, J. Mol. Biol., 403-410) and AlignX for example. At least BLAST, BLAST 2 and FASTA are available for offline and online searching (see Ausubel et al., 1999, pages 7-58 to 7-60), such as for example in the GenomeQuest search tool (www.genomequest.com).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. Vector NTI programs generally use either the public default values or a custom symbol comparison table if supplied (see user manual for further details). For some applications, it is preferred to use the default values for the Vector NTI package.

Alternatively, percentage homologies may be calculated using the multiple alignment feature in Vector NTI (Invitrogen Corp.), based on an algorithm, analogous to CLUSTAL (Higgins D G & Sharp P M (1988), Gene 73(1), 237-244).

Once the software has produced an optimal alignment, it is possible to calculate % homology, preferably % sequence identity. The software typically does this as part of the sequence comparison and generates a numerical result.

Gap Penalties may be used when determining a sequence identity. Examples of parameters used for a pairwise alignment are:

| FOR BLAST | |
|---|---|
| GAP OPEN | 9 |
| GAP EXTENSION | 2 |

| FOR CLUSTAL | DNA | PROTEIN |
|---|---|---|
| Weight Matrix | IUB | Gonnet 250 |
| GAP OPENING | 15 | 10 |
| GAP EXTEND | 6.66 | 0.1 |

In one embodiment, CLUSTAL may be used with the gap penalty and gap extension set as defined above.

Suitably, the degree of identity with regard to a nucleotide sequence is determined over at least 20 contiguous nucleotides, preferably over at least 30 contiguous nucleotides, preferably over at least 40 contiguous nucleotides, preferably over at least 50 contiguous nucleotides, preferably over at least 60 contiguous nucleotides, preferably over at least 100 contiguous nucleotides.

Suitably, the degree of identity with regard to a nucleotide sequence is determined over at least 100 contiguous nucleotides, preferably over at least 200 contiguous nucleotides, preferably over at least 300 contiguous nucleotides, preferably over at least 400 contiguous nucleotides, preferably over at least 500 contiguous nucleotides, preferably over at least 600 contiguous nucleotides, preferably over at least 700 contiguous nucleotides, preferably over at least 800 contiguous nucleotides.

Preferably, the degree of identity with regard to a nucleotide sequence may be determined over the whole sequence, such as over SEQ ID NO:2 or SEQ ID NO: 1 disclosed herein. Suitably, the degree of identity with regard to a protein (amino acid) sequence is determined over at least 100 contiguous amino acids, preferably over at least 200 contiguous amino acids, preferably over at least 300 contiguous amino acids.

Preferably, the degree of identity with regard to an amino acid or protein sequence may be determined over the whole sequence taught herein, such as over SEQ ID NO:2 or SEQ ID NO: 1 disclosed herein.

In the present context, the term “query sequence” means a homologous sequence or a foreign sequence, which is aligned with a subject sequence in order to see if it falls within the scope of the present invention. Accordingly, such query sequence can for example be a prior art sequence or a third party sequence.

In one preferred embodiment, the sequences are aligned by a global alignment program and the sequence identity is calculated by identifying the number of exact matches identified by the program divided by the length of the subject sequence.

In one embodiment, the degree of sequence identity between a query sequence and a subject sequence is determined by 1) aligning the two sequences by any suitable alignment program using the default scoring matrix and default gap penalty, 2) identifying the number of exact matches, where an exact match is where the alignment program has identified an identical amino acid or nucleotide in the two aligned sequences on a given position in the alignment and 3) dividing the number of exact matches with the length of the subject sequence.

In yet a further preferred embodiment, the global alignment program is selected from the group consisting of CLUSTAL and BLAST (preferably BLAST) and the sequence identity is calculated by identifying the number of exact matches identified by the program divided by the length of the subject sequence.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) that may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by synthetic amino acids (e.g. unnatural amino acids) include; alpha* and alpha-disubstituted* amino acids, N-alkyl amino acids*, lactic acid*, halide derivatives of natural amino acids such as trifluorotyrosine*, p-CI-phenylalanine*, p-Br-phenylalanine*, p-I-phenylalanine*, L-allyl-glycine*, ß-alanine*, L-α-amino butyric acid*, L-γ-amino butyric acid*, L-α-amino isobutyric acid*, L-ε-amino caproic acid^{#}, 7-amino heptanoic acid*, L-methionine sulfone^{#}*, L-norleucine*, L-norvaline*, p-nitro-L-phenylalanine*, L-hydroxyproline #, L-thioproline*, methyl derivatives of phenylalanine (Phe) such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid ^{#}and L-Phe (4-benzyl)*. The notation * has been utilised for the purpose of the discussion above (relating to homologous or non-homologous substitution), to indicate the hydrophobic nature of the derivative whereas #has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

The nucleotide sequences for use in the present invention may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences described herein may be modified by any method available in the art. Such modifications may be carried out in order to enhance the in vivo activity or life span of nucleotide sequences of the present invention.

The present invention also encompasses the use of nucleotide sequences that are complementary to the sequences presented herein.

Other variants of the sequences described herein may be obtained for example by probing DNA libraries made from a range of individuals, for example individuals from different populations. In addition, other homologues may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of any one of the sequences in the attached sequence listings under conditions of medium to high stringency. Similar considerations apply to obtaining species homologues and allelic variants of the polypeptide or nucleotide sequences of the invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences.

Alternatively, such polynucleotides may be obtained by site directed mutagenesis of characterised sequences. This may be useful where for example silent codon sequence changes are required to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides.

### Amino Acid Numbering

In the present invention, a specific numbering of amino acid residue positions in the sequences used in the present invention may be employed. By alignment of the amino acid sequence of an candidate autolysin with the autolysin defined in SEQ ID NO:2, it is possible to assign a number to an amino acid residue position in said candidate autolysin which corresponds with the amino acid residue position or numbering of the amino acid sequence shown in SEQ ID NO: 2 of the present invention.

An alternative way of describing the amino acid numbering used in this application is to say that amino acid positions are identified by those ‘corresponding’ to a particular position in the amino acid sequence shown in SEQ ID NO:2. A skilled person will readily appreciate that autolysin sequences vary among different bacterial strains. Reference to the amino acid sequence shown in SEQ ID NO:2 is used merely to enable identification of a particular amino acid location within any particular autolysin protein. Such amino acid locations can be routinely identified using sequence alignment programs, the use of which are well known in the art.

### General Recombinant DNA Methodology Techniques

The present invention employs, unless otherwise indicated, conventional techniques of biochemistry, molecular biology, microbiology and recombinant DNA, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N. Y.); B. Roe, J. Crabtree, and A. Kahn, 1996, DNA Isolation and Sequencing: Essential Techniques, John Wiley & Sons; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

The invention will now be further described by way of Examples, which are meant to serve to assist one of ordinary skill in the art in carrying out the invention and are not intended in any way to limit the scope of the invention.

### Methods to Assay Bacteriophage Sensitivity

As described above, one way to determine the resistance (or sensitivity) to bacteriophage conferred by a modification or a candidate autolysin^{R} allele according to the invention is to determine the resistance of both a bacterial strain sensitive to a phage (such as a P335-like phage, e.g. D6867) (reference strain) and the corresponding derivative bacterial strain in which the candidate modification or autolysin^{R} allele of the invention has been inserted (derivative strain). Thus, the resistance (or sensitivity) to bacteriophage conferred by a modification or candidate autolysin^{R} allele of the invention may be determined by determining the resistance of both a reference strain and the corresponding derivative bacterial strain. The bacteriophage resistance of the two bacterial strains can be determined by calculating the efficiency of plaquing (EOP) in a standard efficiency of plaquing assay, or in the Efficiency of Plaquing Assay I described herein, using a suitable bacteriophage (such as P335-like phage, e.g. D6867) or panel of bacteriophages. The above-mentioned protocol can also be used to screen for candidate autolysin genes of the invention, such as homologues.

The introduction of a modification or of a candidate autolysin^{R} allele of the invention in lieu of or in replacement of the autolysin gene of a bacterial strain sensitive to bacteriophage (such as a P335-like phage, e.g. D6867), using conventional techniques in molecular biology is within the capabilities of a person of ordinary skill in the art. Generally speaking, suitable routine methods include directed mutagenesis or the replacement via homologous recombination of a suitable gene or polynucleotide of interest into the native gene sequence. As detailed above, either a standard efficiency of plaquing assay or the Efficiency of Plaquing Assay I described herein can also be used to determine the bacteriophage resistance (or sensitivity) of a bacterial strain of the invention. The bacteriophage sensitivity of a bacterial strain of the invention can be determined relative to the corresponding bacterial strain which does not comprise the modification, candidate autolysin^{R} allele or polynucleotide of the invention in lieu of the autolysin gene (reference strain).

### Efficiency of Plaquing Assay I

The numeration of infectious phage particles is performed using the double agar overlay plaque assay as described by Kropinski et al. 2009, named “Efficiency of Plaquing Assay I” herein. The method consists of infecting a lawn of bacteria growing on the surface in a soft-agar nutritive medium. Infection by a phage will result in a localized clear or translucent zone corresponding to the area where bacterial are killed or are not growing, termed plaques. The infectious phage unit is thus termed plaque-forming unit (pfu). Because the numeration of phages using the assay necessitates a maximum of 30 to 300 pfu per plate, the phage suspension may require dilution. For this purpose, the primary phage suspension is serially 10-fold diluted in 10 mL of M17 medium containing 5 g/L of lactose (v/v).

The Efficiency of Plaquing Assay I comprises the following steps:
i. pre-cultivate each of the strains to be tested in M17 medium containing 5 g/L of lactose (v/v) overnight at 37° C.;
ii. use each pre-culture separately to seed at 1% (v/v) 5 mL of melted M17-CaCl₂ soft-agar medium containing 5 g/L of lactose, 10 mM of CaCl₂ and 5 g/L (w/v) of agar (that is kept at 47° C. in a water bath);
iii. add 100 μL of the phage dilution to be tested to each of the seeded media;
iv. after mixing, pour each of the mixtures onto the surface of a M17-CaCl₂ solid-agar medium containing 5 g/L of lactose, 10 mM of CaCl₂ and 15 g/L (w/v) of agar;
V. after the solidification of the overlay, incubate the plates inverted for 48 hours at 37° C.; and
vi. enumerate plaques (on plates presenting 30 to 300 plaques).

To calculate the efficiency of plaquing (EOP): calculate the titer of virulent phages as: the number of plaques×10×the reciprocal of the dilution rate; and express in pfu/mL; and calculate the EOP of the phage on a bacterial strain as the titer of the phage on the strain divided by the titer of the phage on the reference strain, and fix the EOP of the reference strain as 1.

Accordingly, the EOP of a bacterial strain which is sensitive to the tested bacteriophage is 1 (i.e. every particle attaching to the host strain can make a plaque on the strain). For example, the EOP of Lactococcus lactis SL12699 with the D6867 phage is 1.

A bacterial strain showing partial sensitivity reduction (or partial resistance) to the tested bacteriophage is characterised by an EOP reduction of at least 4 log, suitably at least 5 log, relative to the reference strain. In one embodiment, a bacterial strain showing partial sensitivity reduction to the tested bacteriophage has an EOP of less than 10⁻⁴, suitably less than 10⁻⁵, relative to the reference strain.

A bacterial strain which has full sensitivity reduction (or is resistant) to the tested bacteriophage is characterised by an EOP reduction of at least 6 log (suitably at least 7 log, suitably at least 8 log, preferably under the detection level of the assay) relative to the reference strain. In one aspect, a bacterial strain which is resistant to the tested bacteriophage has an EOP of less than 10⁻⁶ (suitably less than 10⁻⁷, suitably less than 10⁻⁸) relative to the reference strain. In a preferred aspect, a bacterial strain which is resistant to the tested bacteriophage will provide an EOP of 0 (below detection level, also termed undetectable).

By “reduction of the sensitivity to phage” by EOP Assay I, it is meant an EOP reduction of at least 4 log. In an embodiment, the EOP reduction is of at least 5 log. In an embodiment, the EOP reduction is of at least 6 log. In an embodiment, the EOP reduction is 10 of at least 7 log. In an embodiment, the EOP reduction is of at least 8 log. In an embodiment, a modification or autolysin allele is considered to be a modification or autolysin^{R} allele according to the invention, when the EOP reduction is selected from the group consisting of EOP reduction of at least 4 log, at least 5 log, at least 6 log, at least 7 log and at least 8 log, wherein sensitivity to phage is determined by Efficiency of Plaquing (EOP) Assay I.

Thus, a candidate modification or autolysin allele is considered to be an autolysin^{R} allele according to the invention, when said candidate autolysin allele reduces the sensitivity to phage (such as a P335-like phage) of a Lactococcus lactis SL12699 derivative strain of at least 4 log, said derivative strain being a Lactococcus lactis SL12699 strain into which a modification or autolysiR allele according to the present invention has been introduced and wherein sensitivity to phage (such as P335-like phage) is determined by Efficiency of Plaquing (EOP) Assay I (i.e., as compared to Lactococcus lactis SL12699 strain).

In one aspect, the expression “reduce the sensitivity” or “reduce the EOP’ is defined according to assay I above, i.e., by determining the EOP of phage on the derivative strain and comparing it to the EOP of the same phage DT1 on the parental strain which does not comprise the candidate modification or candidate autolysin^{R} allele.

### EXAMPLES

### Introduction

Lactococcus lactis SL12852 (DGCC12852) was created via phage challenge using parental strain SL12699 (DGCC12699) and P335-like phage D6867. SL12852 is a bacteriophage-insensitive mutant (BIM) against D6867 and acidifies equivalently to SL12699. The mutation responsible for phage resistance in SL12852 was initially unknown. After comparing the genomes of SL12699 and SL12852, differences were found between the two and testing confirmed that a 155-bp deletion in an autolysin gene is responsible for resistance. The wildtype autolysin gene from SL12699 was cloned into pGhost (designated pLys) and electroporated into SL12852. SL12852+pLys isolates became sensitive to D6867. Furthermore, these 12852+pLys isolates were then cured of pLys and gained resistance back to D6867. This suggests that the full wildtype autolysin gene found in SL12699 is essential for certain P335-like phage infection in Lactococcus, which is a gene that has not yet been shown to be associated with phage resistance.

### Materials and Methods

### DNA Isolation, Sequencing, and Bioinformatics Analysis

High titer lysates of D6867 and D7138 were purified using PureLink Viral RNA/DNA Mini Kit (Thermo Fisher Scientific, Waltham, MA, USA). Concentration of the DNA sample was measured using the Invitrogen Qubit. Each sample was then run on a gel to confirm amplicons. The sample was sent to University of Illinois Urbana-Champaign for Nanopore sequencing and genome assembly. Phage genomes were compared to genomes of known phage types from DuPont's Culture Development Genome Database. Comparisons were done on Geneious 11.0.5 using the Mauve genome alignment viewer.

Isolated colonies of SL12699 and SL12852 were grown overnight in M17-L broth. DNA preparation was performed using MasterPure Complete DNA and RNA Purification Kit (Lucigen Corp., Middleton, WI, USA). From the protocol, “Cell Samples” “Precipitation of DNA” were followed; amounts of reagents used were multiplied by 10. Nanopore and Illumina sequencing was done at University of Illinois Urbana-Champaign. SL12699 and SL12852 genome comparisons were done on Geneious 11.0.5 using the Mauve genome alignment viewer. The areas of the chromosome in which differences were found were subsequently Sanger sequenced to confirm/refute the mutations by designing primers specific to that area. DNA from SL12852 and other BIMs were spotted onto FTA (Flinder's Technology Association) paper and cleaned according to manufacturer's instructions. PCR was performed with Phusion® High-Fidelity PCR Master Mix with HF Buffer (New England Biolabs Inc., Upswich, MA, USA); primers were designed from Integrated DNA Technologies. PCR products were prepared for sequencing using Wizard SV Gel and PCR Clean-Up System (Promega Corp., Madison, WI, USA). Sequencing was performed at Eurofins Scientific (Louisville, KY, USA) and results were analysed using the Map to Reference tool on Geneious 11.0.5.

### Autolysin Complementation Assay

Mutant SL12852 was complemented with the wildtype autolysin. First, the wildtype autolysin was cloned into pGhost9 (see Table 1) using Gibson assembly. The vector pGhost9 was linearized with primers VF-pg9 and VR-pg9. The insert was amplified with the primers CloneAutoF and CloneAutoR with Phusion polymerase. Both primers had ˜20-nucleotide extensions complementing the 3′- and 5′-ends, respectively, of the linearized vector. Purified amplicons were assembled using NEBuilder HiFi DNA Assembly MasterMix (New England Biolabs, Upswich, MA, USA) according to manufacturer's instructions. Recombinant vector pLys was assembled in TG1RepA and then purified from TG1RepA using the GeneJET Plasmid Miniprep Kit (Thermo Scientific, USA). Electroporation of L. lactis was performed as previously described (Holo and Nes, 1989 Appl. Environ. Microbiol. 55:3119-3123). Transformants were PCRd with primers RSF and GC pG9vF to check for presence of pGhost9 containing the autolysin.

pLys was cured from SL12852 transformants by growing in milk at 37° C. in the absence of erythromycin for 3 consecutive nights. Single colony isolates were tested for loss of erythromycin resistance. PCR was performed with primers RSF and GC pG9vF to confirm absence of the plasmid. Sanger sequencing was done using primers Autolysin_F1, Autolysin_F2, and Autolysin_F3 to confirm the chromosomal autolysin is still truncated; initial amplification was completed using primers AutolysinNested_F and AutolysinNested_R. See Table 2 for primer specifications.

TABLE 1
Bacteria, phages, and plasmids used in this study.

| | Biological | Relevant | |
|---|---|---|---|
| | Material | Characteristics | Reference |
| Bacteria | SL12699 | Lactococcus lactis ssp. | Deposited at the Leibniz Institute |
| | (=DGCC12699) | lactis; phage-sensitive | DSMZ-German Collection of |
| | | starter strain | Microorganisms and Cell |
| | | | Cultures on 4 Aug. 2020; |
| | | | Accession number given by the |
| | | | International Depositary |
| | | | Authority: DSM 3360 |
| | SL12852 | Lactococcus lactis ssp. | DuPont collection. Natural |
| | (=DGCC12852) | lactis; phage-resistant | phage-resistant mutant of |
| | | starter strain | SL12699 generated by |
| | | | challenge with phage D6867. |
| | TG1RepA | Escherichia coli; host for | Duwat, et al., 1997. J Bacteriol. |
| | | plasmid propagation | 179: 4473-4479. |
| Phage | D6867 | P335-like; infects host | Deposited at the Leibniz Institute |
| | | SL12699 | DSMZ-German Collection of |
| | | | Microorganisms and Cell |
| | | | Cultures on 4 Aug. 2020; |
| | | | Accession number given by the |
| | | | International Depositary |
| | | | Authority: DSM 33596 |
| | D7138 | P335-like; infects host | DuPont collection. |
| | | SL12699 | |
| Plasmids | pGhost9 | Erythromycin-resistant | Maguin et al., 1996 J. Bacteriol. |
| | | vector for insertional | 178: 931-935. |
| | | mutagenesis with a | |
| | | temperature-sensitive | |
| | | origin of replication | |
| | pLys | pGhost9 with the | This study. |
| | | wildtype autolysin | |
| | | incorporated from | |
| | | SL12699 | |

TABLE 2
Primers used in this study.

| Primer Name | Sequence (5′-3′) | Target |
|---|---|---|
| CloneAutoF | TTCTGGAATGGGATTCAGGACTACCACTATTCCCAAAG | Autolysin gene |
| | CATTTC (SEQ ID NO: 3) | incorporated in |
| | | pGhost9 |
| CloneAutoR | ATCCGTGTCGTTCTGTCCACGCAATGGGAGATGATTGG | Autolysin gene |
| | AGAGG (SEQ ID NO: 4) | incorporated in |
| | | pGhost9 |
| VF-pg9 | GTGGACAGAACGACACGGAT (SEQ ID NO: 5) | pGhost9 |
| VR-pg9 | TCCTGAATCCCATTCCAGAA (SEQ ID NO: 6) | pGhost9 |
| RSF | TTCTGGAATGGGATTCAGGA (SEQ ID NO: 7) | pGhost9 |
| GC pG9vF | ATCCGTGTCGTTCTGTCCAC (SEQ ID NO: 8) | pGhost9 |
| Autolysin_F1 | CTCACATTATTTCACTAATACTCTTTTCTGG | Autolysin gene |
| | (SEQ ID NO: 9) | |
| Autolysin_F2 | CATGGATTGGAACAGATACAGCAACTTG | Autolysin gene |
| | (SEQ ID NO: | |
| Autolysin_F3 | CAATACGATCTATACTACCGAGTTCAAGC | Autolysin gene |
| | (SEQ ID NO: 11) | |
| internalErmR-F | GCTGAATCGAGACTTGAGTG (SEQ ID NO: 12) | EmR in |
| internalErmR-R | GTCATCTATTCAACTTATCG (SEQ ID NO: 13) | EmR in |
| Autolysin | GCCAACTTTGTCTTCATACTCACC (SEQ ID NO: 14) | Autolysin gene |
| Nested_F | | |
| AutolysinNested_ | GTATGTTTTGGAAGCCCTCAGG (SEQ ID NO: 15) | Autolysin gene |

### Testing Milk Acidification Rates

For initial assessment of acidification properties, overnight 11% NFDM BIM growths were transferred into M17-Lac. After the cultures reached an OD₆₀₀˜0.65, they were inoculated at 0.75% into activity milk (commercial 1% fat Kemp's). Samples were kept in a 30° C. water bath overnight with pH probes and Cinac software monitoring acidification of each strain every 2 minutes. Each strain was run in duplicate.

### Results

### SL12699 and SL12852 Genome Results

After comparing SL12699 and SL12852 genomes on Geneious, a 153-bp deletion was found at position 1,225 of an autolysin gene. This mutation does not result in an early stop codon but truncates the second half of the protein by 51 amino acids. This mutation was confirmed with Sanger sequencing.

### D6867 and D7138 Genome Results

Upon comparing the D6867 and D7138 genomes to known phage types, several areas of the genomes showed matches to P335-like phages; D6867 and D7138 show highest similarity to a phage classified as smq86 type.

Additionally, genomes D6867 and D7138 revealed that both phages have high similarity to a prophage in SL12699 (95.1% and 95.8% pairwise identity, respectively). This suggests that the phages are excised prophages originating from SL12699.

### Sanger Sequencing Results

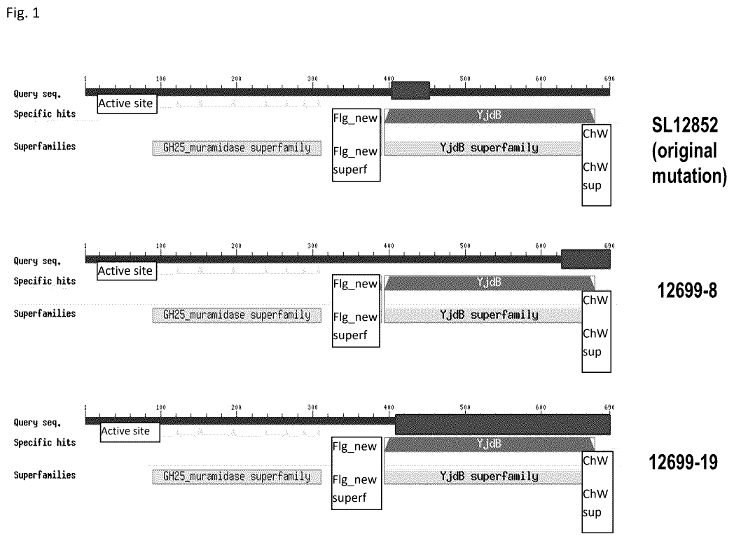

Sanger sequencing was done on four additional BIMs of SL12699. Results showed two of the BIMs to have the same 153-bp deletion found in SL12852. BIM 8 showed a G-deletion at nucleotide 1,805-1,809 in a run of G's; this deletion causes a frameshift which results in a premature stop codon at position 1,880. BIM 19 showed a G to A SNP at nucleotide 1,229, which changes that same codon to an early stop codon. FIG. 1 shows the three different mutations found in the autolysin; all of these mutations are located within the yjdB domain of the protein.

SL12852 pLys Transformants

Ten SL12852+pLys isolates and two SL12852+pGhost isolates were plated and spotted with D6867 and D7138 to check phage sensitivity. Results showed that after adding pLys to SL12852, the isolates became fully sensitive to both phages. Additionally, results showed that pGhost9 alone does not affect phage sensitivity in SL12852 (see Table 3).

TABLE 3
D6867 and D7138 phage sensitivity results of
SL12852 + pLys and SL12852 + pGhost isolates
displayed as EOPs (efficiency of plaquing).

EOPs

| Strain | D6867 | D7138 |
|---|---|---|
| SL12699 | 1 | 1 |
| SL12852 + pLys (1) | 0.978 | 1.036 |
| SL12852 + pLys (2) | 1.130 | 1.066 |
| SL12852 + pLys (3) | 1.108 | 1.006 |
| SL12852 + pLys (4) | 0.956 | 1.012 |
| SL12852 + pLys (5) | 1.043 | 0.964 |
| SL12852 + pLys (6) | 0.956 | 1.018 |
| SL12852 + pLys (7) | 1.347 | 1.024 |
| SL12852 + pLys (8) | 0.978 | 0.970 |
| SL12852 + pLys (9) | 1.152 | 1.054 |
| SL12852 + pLys (10) | 1.043 | 1.072 |
| SL12852 + pGhost* | <4.6E− | <1.7E− |
| SL12852 + pGhost (2) | <4.6E− | <1.7E− |

SL12699 was used as the positive (phage-sensitive) control.
*This EOP reduction shows that the vector alone does not provide any phage sensitivity (since SL12852 is already naturally resistant).

pLys Curing in SL12852

After 37° C. propagation, five SL12852+pLys samples were plated on both SR Lac and SR Lac+Erm media. Their inability to grow on Erm media indicated loss of pLys. Subsequent colony PCR of isolates picked from SR Lac plates confirmed loss of pLys, referred to as SL12852 (−) pLys. Each SL12852 (−) pLys isolate was plated and spotted with D6867 and D7138. Results show that they were fully phage resistant which further confirms that the complemented wildtype autolysin is required for phage sensitivity (see Table 4).

TABLE 4
D6867 and D7138 phage sensitivity results
of SL12852 (—) pLys colonies.

| | |
|---|---|
| D6867 | D7138 |

| | 0 | −1 | 0 | −1 |
|---|---|---|---|---|
| SL12699 | Clear | Clear | Clear | Clear |
| SL12852 (—) | Lawn | Lawn | Lawn | Lawn |
| SL12852 (—) | Lawn | Lawn | Lawn | Lawn |
| SL12852 (—) | Lawn | Lawn | Lawn | Lawn |
| SL12852 (—) | Lawn | Lawn | Lawn | Lawn |
| SL12852 (—) | Lawn | Lawn | Lawn | Lawn |

A clearing or isolated plaques in the lawn of bacteria indicates phage sensitivity; an uninterrupted lawn indicates resistance.

### Acidification and Phage Robustness

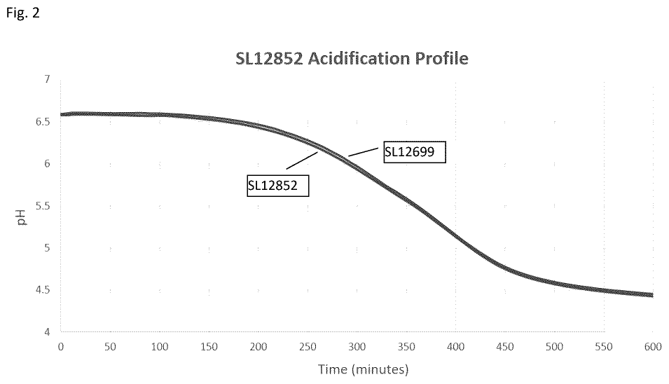

The milk acidification properties of SL12852 were tested. Cinac activity curves show that SL12852 acidified equivalently to the parent (see FIG. 2).

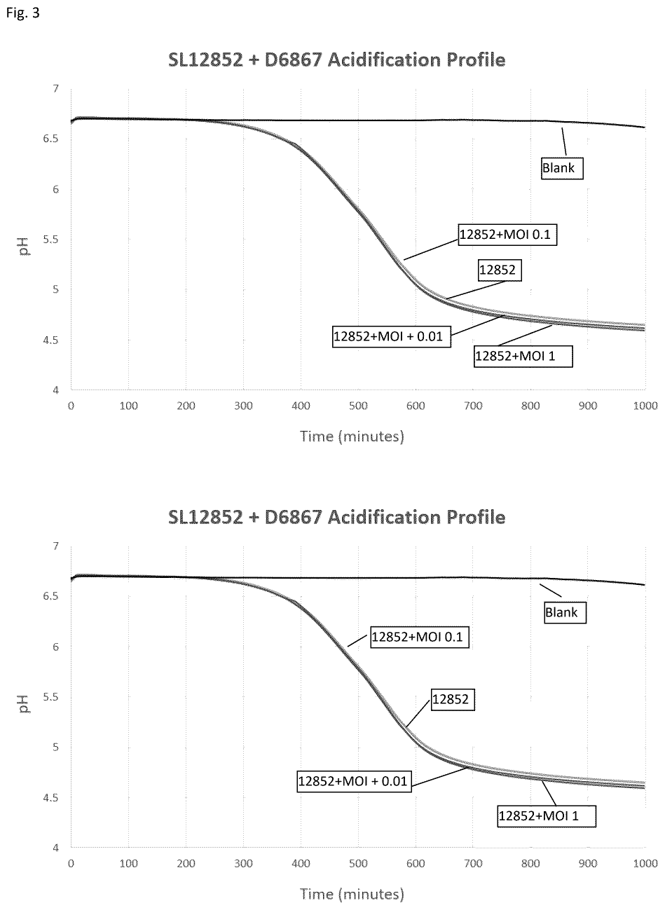

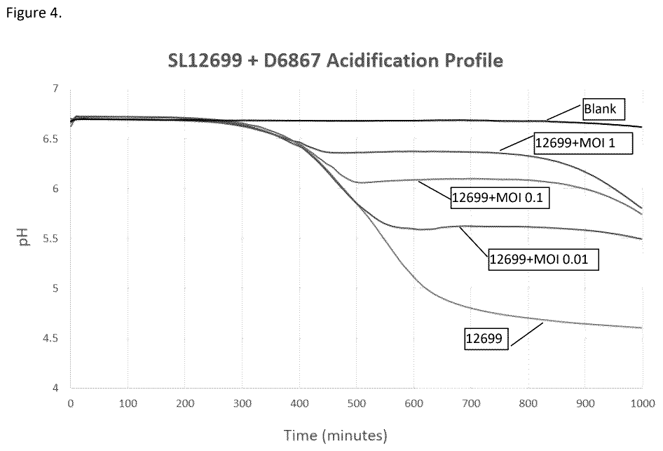

In secondary assays, acidification rates of SL12852 were tested in the presence of phage D6867 at varying multiplicities of infection (0.01, 0.1, and 1). Parallel assays of parent SL12699 served as phage controls (see FIGS. 3 and 4). Results show that even with the highest MOI of 1 added, there is still no slowdown in acidification of SL12852, indicating a strong level of phage resistance.

### Discussion

Phage are commonly present and problematic for starter strains used in industrial dairy fermentations; P335 type are particularly problematic. For P335-like phage, a single gene or protein has not yet been proven to be essential for successful P335-like phage infection.

In this study, truncation of the autolysin gene found in SL12699 results in complete resistance against P335-like phage, and therefore, represents a unique genetic target to address issues for this class of phage.

In this project, phage-resistant strain SL12852 was electroporated with a plasmid containing the wildtype autolysin gene, which resulted in the isolates becoming fully sensitive to P335-like phage D6867. Furthermore, when this plasmid was taken out of those isolates, the strain become fully resistant to D6867 again. PCR amplification using primers specific to that plasmid was used in confirming presence/absence for the wildtype autolysin. Additionally, plasmid profiling confirmed both sets of resultant isolates to be derived from SL12852. Lastly, Sanger sequencing confirmed that the chromosomal autolysin remained truncated throughout this experiment.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A bacterial strain which has been modified to reduce the activity and/or expression of autolysin, wherein said modified strain has reduced sensitivity to bacteriophage.

2. A bacterial strain according to claim 1, where said modification renders autolysin partially or completely non-functional with respect to phage infection.

3. A bacterial strain according to claim 1, wherein said modification is a stop codon, an insertion, a deletion or a mutation.

4. A bacterial strain according to claim 1, where said modification has been introduced into a nucleic acid sequence which encodes said autolysin, or in to a regulatory region which contributes to controlling the expression of said autolysin.

5. A bacterial strain according to claim 1, wherein said strain has been modified to comprise an autolysin^{R} allele encoding an autolysin^{R} protein, wherein said autolysin^{R} allele reduces the sensitivity to phage D6867 of a Lactococcus lactis SL12699 derivative strain, said SL12699 derivative strain being a Lactococcus lactis SL12699 strain into which its autolysin allele has been replaced by said autolysin^{R} allele, and wherein sensitivity to phage D6867 is determined by Efficiency of Plaquing (EOP) Assay I.

6. The bacterial strain according to claim 1, wherein said strain is homozygous for the modification.

7. A method for increasing the resistance of a bacterial strain to bacteriophage comprising modifying said bacterial strain by decreasing the activity and or expression of autolysin.

8. A method for preparing at least one bacteriophage resistant variant strain, comprising modifying a parental bacterial strain to decrease the activity and or expression of autolysin.

9. A method according to claim 7 comprising:
a) providing a bacterial strain sensitive to at least one bacteriophage;
b) modifying said bacterial strain to reduce the activity and/or expression of autolysin, wherein said modified strain has reduced sensitivity to bacteriophage; and
c) recovering the strain(s) having reduced sensitivity to at least one bacteriophage.

10. A bacterial strain obtainable or obtained by the method of claim 7.

11-14. (canceled)

15. The bacterial strain according to claim 1, wherein reduction of sensitivity to phage is characterized by an EOP reduction of at least 4 log, of at least 5 log or of at least 6 log.

16. The bacterial strain according strain according to claim 1, wherein said modification encodes an autolysin^{R} protein comprising an amino acid suppression, an amino acid addition, an amino acid substitution or an amino acid suppression and addition, relative to an autolysin protein selected from the group consisting of:
a) an autolysin protein having an amino acid sequence as defined in SEQ ID NO:2;
b) an autolysin variant protein comprising an amino acid sequence having at least 80% identity with SEQ ID NO:2 encoded by a autolysin allele, which does not reduce the EOP of phage D6867 on a Lactococcus lactis SL12699 derivative strain, said SL12699 derivative strain being a Lactococcus lactis SL12699 strain into which its autolysin allele has been replaced by the autolysin allele encoding said autolysin variant protein and wherein sensitivity to phage D6867 is determined by Efficiency of Plaquing (EOP) Assay I; and
c) an autolysin variant protein comprising an amino acid sequence having at least 80% identity with SEQ ID NO:2 encoded by a autolysin allele, which reduces the EOP of phage D6867 on a Lactococcus lactis SL12699 derivative strain, of less than 3 log, said D6867 derivative strain being a Lactococcus lactis SL12699 strain into which its autolysin allele has been replaced by the autolysin allele encoding said autolysin variant protein and wherein sensitivity to phage DT1 is determined by Efficiency of Plaquing (EOP) Assay I.
d) a homologue of SEQ ID NO:2 encoded by a autolysin allele, which does not reduce the EOP of phage D6867 on a Lactococcus lactis SL12699 derivative strain, said SL12699 derivative strain being a Lactococcus lactis SL12699 strain into which its autolysin allele has been replaced by the autolysin allele encoding said autolysin variant protein and wherein sensitivity to phage D6867 is determined by Efficiency of Plaquing (EOP) Assay I; and
e) a homologue of SEQ ID NO:2 encoded by a autolysin allele, which reduces the EOP of phage D6867 on a Lactococcus lactis SL12699 derivative strain, of less than 3 log, said D6867 derivative strain being a Lactococcus lactis SL12699 strain into which its autolysin allele has been replaced by the autolysin allele encoding said autolysin variant protein and wherein sensitivity to phage DT1 is determined by Efficiency of Plaquing (EOP) Assay I.

17. The bacterial strain according strain according to claim 1, wherein said modification results in truncation of the YjdB domain of autolysin.

18. The bacterial strain according to claim 1, wherein said truncated autolysin protein is the result of a deletion and/or an insertion in the autolysin allele.

19. The bacterial strain according to claim 1, wherein said truncated autolysin protein is the result of a nucleotide mutation leading to a STOP codon.

20. The bacterial strain according to claim 1, wherein said strain is additionally mutated in its pip gene and resistant to c2-type c2 phages.

21. The bacterial according to claim 1, wherein said strain is additionally mutated in its yjaE gene and resistant to bil67-type c2 phages.

22. The bacterial strain according to claim 1, wherein said strain comprises a lactic acid strain.

23. (canceled)

24. A food or feed product comprising the strain of claim 1.

25. (canceled)

26. A method for manufacturing a fermented product, comprising:
a) inoculating a substrate with the strain of claim 1; and
b) fermenting the inoculated substrate obtained from step a) to obtain a fermented product.

27. (canceled)


SEQUENCE LISTING (15 sequences)

SEQ ID NO: 1 - DNA, 2073 bp, Artificial Sequence
  nucleotide sequence encoding SL12699 autolysin
atgaaactgaaaaaaactcacattatttcactaatactcttttctggattactatttacaaaaccagtattagctgaagacactattagttcagataacaccccgatggggcactacattgagcaaattcaaactcaagaaacttccaaggacagtagtagtgatacatcatctacaactcctaatatcagagcacgttcatttgcagcagctccaactgccaatgttccctctgatctcaagtccgatgataatacactgccaagaaaagatgctgtagatattgctagctatcaatcatggatgacacaagcagactttaattctcttaagacatctggtgtcaagtcaatcgttgttaaactaaccgaaggaactaattatacgaacccatatgctgcaaatcaaattaaaatggcacagaatgccggcttaaatgttgcagtttatcactacgcacgcttaacaggagccaattcacaaagtgatgcaaattcattagccatacaggaagccgcatactttgcaaaagtagctaaatcttttggattgtctagcaacattgtaatgattatggactgcgagcaaccttatagagatggctcgggaaatattatcggacccaatcccataacggttgattgggcaacagctggtgtacaatttgctaacacgctcaaagcaaatggttatagcaacacaaagttttatacctctgcttcatggattggaacagatacagcaacttgtcaaatgaactataatactctaggtggtctcaaaaatctttgggctgctcaatatctatatggcaagccatcttctagtaatcttcagaatacgcaatatggtgcttggcaatatacaagtcagatgtattatcaaggaacctctaacttgaaagccaatgctgttgatacttcaattgattatagtaattattttacctctacatcccctgttcctccttcaacttatactataacatttaacacagacggtggaacaccaattgccaatcaatcggttgcagctggtagtgtagtaagccaaccagcagctcctactaaagctggatttaacttctcaggttggtatagtgattcttcacttactcaagcttataacttcgcaaaagtaattacatctaatacaaccctttatgctaaatggatcccgataacatctccttcaatttcttaccaagcccaagtccaaaatataggatggcaaaaaaattcctataacggagaaactgctggtacaactgggttagggttacgcatggagtcacttaaaattagccttatcaacctttcaaacggtctgactaattcaaatagtcacattcaatatcagggttatgtacaaaatatcggatggcaaaatccggttcaagatgggacaattgctggaacagttggacaagggttgcgatttgaggcaataaaaatgaatttaagcggagaaatagcaaatcaatacgatctatactaccgagttcaagctcaaaatattggatggatggattgggcaaaaaatggcgaagctgctggaacttctacgatgtcctaccgtttagaagccattcaaattcaactcgttaaaaaaggaaatccagctcctggagctacaacttttacttttttgactactccttccctccagtattgcactcaggttcaaaacattggctggcaaaatcctgtatctactggtcagatctctggaacggtgggtaaaagcttacgaacggaagctttaaaaattaatattggcaacctttcagcaggtattgacggtggggtatcgtatagcagccaaattcaaaatattggctggcaagcaccagtatccaatggtcaaatctctggaactgtaggacaaagtctacgtttagaggccttgaaaataaacctcactggtagaatctcacagtattttgatatcaattatgccagccaagtccaaaacatcggctggcaagcaccagtatccaacggtcaaatctctggaactgtgggaatgtccttacgagttgaagctgtaaaactctctctttctccaaaataa

SEQ ID NO: 2 - protein, 690 aa, Artificial Sequence
  SL12699 autolysin amino acid sequence
MKLKKTHIISLILFSGLLFTKPVLAEDTISSDNTPMGHYIEQIQTQETSKDSSSDTSSTTPNIRARSFAAAPTANVPSDLKSDDNTLPRKDAVDIASYQSWMTQADFNSLKTSGVKSIVVKLTEGTNYTNPYAANQIKMAQNAGLNVAVYHYARLTGANSQSDANSLAIQEAAYFAKVAKSFGLSSNIVMIMDCEQPYRDGSGNIIGPNPITVDWATAGVQFANTLKANGYSNTKFYTSASWIGTDTATCQMNYNTLGGLKNLWAAQYLYGKPSSSNLQNTQYGAWQYTSQMYYQGTSNLKANAVDTSIDYSNYFTSTSPVPPSTYTITFNTDGGTPIANQSVAAGSVVSQPAAPTKAGFNFSGWYSDSSLTQAYNFAKVITSNTTLYAKWIPITSPSISYQAQVQNIGWQKNSYNGETAGTTGLGLRMESLKISLINLSNGLTNSNSHIQYQGYVQNIGWQNPVQDGTIAGTVGQGLRFEAIKMNLSGEIANQYDLYYRVQAQNIGWMDWAKNGEAAGTSTMSYRLEAIQIQLVKKGNPAPGATTFTFLTTPSLQYCTQVQNIGWQNPVSTGQISGTVGKSLRTEALKINIGNLSAGIDGGVSYSSQIQNIGWQAPVSNGQISGTVGQSLRLEALKINLTGRISQYFDINYASQVQNIGWQAPVSNGQISGTVGMSLRVEAVKLSLSPK

SEQ ID NO: 3 - DNA, 44 bp, Artificial Sequence
  primer CloneAutoF
ttctggaatgggattcaggactaccactattcccaaagcatttc

SEQ ID NO: 4 - DNA, 43 bp, Artificial Sequence
  primer CloneAutoR
atccgtgtcgttctgtccacgcaatgggagatgattggagagg

SEQ ID NO: 5 - DNA, 20 bp, Artificial Sequence
  primer VF-pg9
gtggacagaacgacacggat

SEQ ID NO: 6 - DNA, 20 bp, Artificial Sequence
  primer VR-pg9
tcctgaatcccattccagaa

SEQ ID NO: 7 - DNA, 20 bp, Artificial Sequence
  primer RSF
ttctggaatgggattcagga

SEQ ID NO: 8 - DNA, 20 bp, Artificial Sequence
  primer GC pG9vF
atccgtgtcgttctgtccac

SEQ ID NO: 9 - DNA, 31 bp, Artificial Sequence
  primer Autolysin_F1
ctcacattatttcactaatactcttttctgg

SEQ ID NO: 10 - DNA, 28 bp, Artificial Sequence
  primer Autolysin_F2
catggattggaacagatacagcaacttg

SEQ ID NO: 11 - DNA, 29 bp, Artificial Sequence
  primer Autolysin_F3
caatacgatctatactaccgagttcaagc

SEQ ID NO: 12 - DNA, 20 bp, Artificial Sequence
  primer internalErmR-F
gctgaatcgagacttgagtg

SEQ ID NO: 13 - DNA, 20 bp, Artificial Sequence
  primer internalErmR-R
gtcatctattcaacttatcg

SEQ ID NO: 14 - DNA, 24 bp, Artificial Sequence
  primer AutolysinNested_F
gccaactttgtcttcatactcacc

SEQ ID NO: 15 - DNA, 22 bp, Artificial Sequence
  primer AutolysinNested_R
gtatgttttggaagccctcagg